(19) United States
(12) Patent Application Publication
(10) Pub. No.: US 2010/0151019 A1
(43) Pub. Date: Jun. 17, 2010

(54) SOLID COMPOSITION FOR CONTROLLED RELEASE OF IONIZABLE ACTIVE AGENTS WITH POOR AQUEOUS SOLUBILITY AT LOW pH AND METHODS OF USE THEREOF

(75) Inventors: Chandir Ramani, Brampton (CA); Juan Wang, Foster City, CA (US); Anil Kane, Mississauga (CA); Kwok Chow, Mississauga (CA); Joe Lambing, South San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Portola Pharmaceuticals, Inc., South San Francisco, CA (US)

(21) Appl. No.: 12/618,511

(22) Filed: Nov. 13, 2009

Related U.S. Application Data
(60) Provisional application No. 61/115,008, filed on Nov. 14, 2008.
(60) Provisional application No. 61/114,941, filed on Nov. 14, 2008.

(51) Int. Cl. A61K 9/22; A61K 31/517; A61P 9/00; A61P 7/02

(52) U.S. Cl. 424/465; 514/266.24

(57) ABSTRACT

A novel solid composition and methods for making and using the solid composition are provided. The solid composition comprises: (a) at least one active agent with a solubility of less than about 0.3 mg/ml in an aqueous solution with a pH of at most about 6.8 at a temperature of about 37° C.; and (b) a hydrophilic polymer matrix composition comprising: i) a hydrophilic polymer selected from the group consisting of METHOCEL™, POLYOX™ WSR 1105 and combinations thereof; and optionally ii) a hydrophobic polymer selected from the group consisting of Ethocel 20 premium; and (c) an alkalizer selected from the group consisting of calcium carbonate, magnesium oxide heavy and sodium bicarbonate; wherein the composition provides at least about 70% release of the active between about 7 to about 12 hours following oral administration.

### CROSS REFERENCES TO RELATED APPLICATIONS

This application claims the benefit of U.S. Provisional Patent Application Nos. 61/115,008 filed Nov. 14, 2008 and 61/114,941 filed Nov. 14, 2008, the disclosures of each of which are hereby incorporated by reference in their entirety for all purposes.

### TECHNICAL FIELD OF INVENTION

The present invention relates to the field of pharmaceutical formulations and the methods for optimizing drug absorption rate of orally administered, weakly acidic drugs, or their pharmaceutically acceptable salts with poor or reduced aqueous solubility. More particularly, the present invention concerns a formulation comprising an active in a controlled release tablet formulation for the treatment for thrombotic complications.

### BACKGROUND OF THE INVENTION

Compounds having the formula (I):

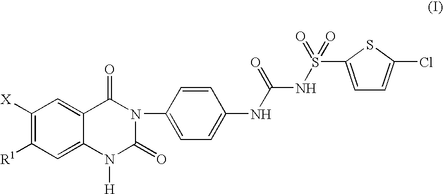

wherein: R¹ is selected from the group consisting of H, halogen, —OH, —C₁₋₁₀-alkyl and C₁₋₆-alkylamino; and X is selected from the group consisting of: F and I; for example, are being developed for the treatment of thrombotic complications. [4-(6-fluoro-7-methylamino-2,4-dioxo-1,4-dihydro-2H-quinazolin-3-yl)-phenyl]-5-chloro-thiophen-2-yl-sulfonylurea potassium salt (Compound 1) has a molecular weight of 562.04 (free acid 523.95). Its pKa is about 3.3 with a logP of about 2.5 and logD (pH7.4) of about −1.6. The aqueous solubility of the free acid form is <0.1 mg/ml (i.e. practically insoluble) at pH 1.0-7.4.

Compounds of formula (I) have been shown to be platelet ADP receptor inhibitors and accordingly, are useful in the prevention and/or treatment of cardiovascular diseases, particularly those related to thrombosis.

Thrombotic complications are a major cause of death in the industrialized world. Examples of these complications include acute myocardial infarction, unstable angina, chronic stable angina, transient ischemic attacks, strokes, peripheral vascular disease, preeclampsia/eclampsia, deep venous thrombosis, embolism, disseminated intravascular coagulation and thrombotic cytopenic purpura. Thrombotic and restenotic complications also occur following invasive procedures, e.g., angioplasty, carotid endarterectomy, post CABG (coronary artery bypass graft) surgery, vascular graft surgery, stent placements and insertion of endovascular devices and prostheses, and hypercoagulable states related to genetic predisposition or cancers. It is generally thought that platelet aggregates play a critical role in these events. Blood platelets, which normally circulate freely in the vasculature, become activated and aggregate to form a thrombus from disturbed blood flow caused by ruptured atherosclerotic lesions or by invasive treatments such as angioplasty, resulting in vascular occlusion. Platelet activation can be initiated by a variety of agents, e.g., exposed subendothelial matrix molecules such as collagen, or by thrombin which is formed in the coagulation cascade.

Indomethacin, 2-{1-[(4-chlorophenyl)carbonyl]-5-methoxy-2-methyl-1H-indol-3-yl}acetic acid, has the formula:

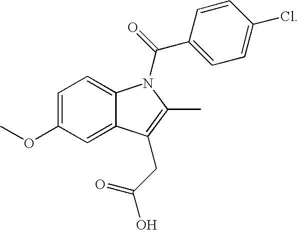

Indomethacin has a molecular weight of 357.787. Its pKa is about 4.5 with a logP of about 3.8 and logD (pH7.4) of about 0.30 (International Journal of Pharmaceutics Volume 193, Issue 2, 5 Jan. 2000, Pages 261-264). The aqueous solubility of the free acid form is less than about 0.25 mg/ml (i.e. practically insoluble) at pH 1.0-7.4. Indomethacin is a non-steroidal anti-inflammatory drug commonly used to treat conditions such as, fever, pain, stiffness, and swelling. It works by inhibiting the production of prostaglandins, which cause these symptoms.

Ketoprofen, (RS)-2-(3-benzoylphenyl)propanoic acid, has the formula:

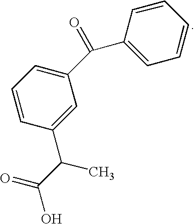

Ketoprofen has a molecular weight of 254.281. Its pKa is about 5.94 with a logP of about 0.97, and logD (pH7.4) of about 1.34. The aqueous solubility of the free acid form is less than about 0.2 mg/ml (i.e. practically insoluble) at pH 1.0-7.4. Ketoprofen is one of the propionic acid class of non-steroidal anti-inflammatory drugs (NSAID) with analgesic and antipyretic effects. It also acts by inhibiting the production of prostaglandin.

Naproxen, (+)-(S)-2-(6-methoxynaphthalen-2-yl) propanoic acid, has the formula:

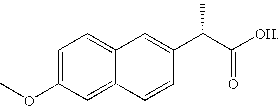

Naproxen has a molecular weight of 230.259. Its pKa is about 4.2 with a logP of about 3.22 and logD (pH7.4) of about 0.79. The aqueous solubility of the free acid form is <0.1 mg/ml (i.e. practically insoluble) at pH 1.0-7.4. Naproxen Sodium is also one of the propionic acid class of NSAIDs commonly used for the reduction of mild to moderate pain, fever, inflammation and stiffness caused by conditions such as osteoarthritis, rheumatoid arthritis, psoriatic arthritis, gout, ankylosing spondylitis, menstrual cramps, tendinitis, bursitis, and the treatment of primary dysmenorrhea. It works by inhibiting both the COX-1 and COX-2 enzymes.

Many therapeutically active, acid compounds, including those described above, have a very narrow absorption window and are absorbed only in upper part of small intestine and not or minimally absorbed in the colonic region. These compounds can also be very sensitive to moisture degradation. Therefore a challenge in formulating these compounds is to release the drug in the stomach and upper GI tract (e.g. duodenum), where the drug is well absorbed, when the drug is poorly soluble at acidic pHs in the stomach and upper GI tract.

Techniques have been disclosed for preparing sustained (or controlled) release pharmaceutical formulations and gastric retentive properties. The limitations associated with these prior art dosage forms is that they do not provide consistent release profiles for pH-solubility dependent drug and do not provide zero order release profiles.

There exists a continuing need for further improvement in pharmaceutical preparations with a controlled release profile for a gastro-retentive type solid composition containing compounds with poor aqueous solubility, such as Compound 1, and other weakly acidic drugs, or their pharmaceutically acceptable salts. Specifically, there remains a need for a tablet to stay in the upper GI tract and/or release a weakly acidic drug or a pharmaceutically acceptable salt thereof, such as Compound 1 for 7-9 hours (fast release (FR) or a 10-12 hours (slow release: SR) in its dissolved (ionized) form from the formulation in a stable manner independent of the pH of the stomach, that would allow the use of a medicine in a once or twice-a-day regime. The present invention satisfies these and other needs.

### BRIEF SUMMARY OF THE INVENTION

The present inventors conceived that the decrease in bioavailability of a weakly acidic drug compound or a pharmaceutically acceptable salt thereof, with poor aqueous solubility, such as Compound 1, could be improved by providing an alkaline environment for Compound 1 while releasing the drug from a matrix system as it is exposed to and hydrated with acidic environment in the stomach after oral administration, and conducted extensive studies thereon. As a result, the present inventors developed orally administrable pharmaceutical compositions and methods which can improve the bioavailability of a weakly acidic drug, or a pharmaceutically acceptable salt thereof, such as Compound 1, by releasing the drug for 7-9 hours (fast release: FR) or 10-12 or 24 hours (slow release: SR), and thus, completed the present invention.

Therefore, a purpose of the present invention is to provide orally administrable pharmaceutical compositions for improving the bioavailability and/or reducing the dosing intervals of a drug with poor aqueous solubility. Another purpose of the present invention is to provide methods for improving the bioavailability of an orally administered drug and methods of producing such solid formulations.

The present invention is applicable not only to ADP receptor antagonists but also to other weakly acidic drugs with poor aqueous solubility.

One aspect of the present invention relates to a solid pharmaceutical composition for the controlled release of an active agent in the gastrointestinal tract, comprising:
(a) at least one weak acid active agent with a solubility of less than about 0.3 mg/ml in an aqueous solution at a pH of at most about the pKa of the active acid at a temperature of about 25 to about 37° C., or a pharmaceutically acceptable salt thereof;
(b) at least one hydrophilic polymer which is not instantly soluble in gastric fluids; and
(c) at least one alkalizer;
wherein the composition reduces evacuation from the stomach; and
   provides at least about 70% release of the active for a period of time between about 7 to about 12 hours following oral administration.

In another aspect of the present invention relates to a solid pharmaceutical composition for the controlled release of an active agent in the gastrointestinal tract, comprising:
(a) at least one weak acid active agent with a solubility of less than about 0.2 mg/ml in an aqueous solution at a pH of at most about the pKa of the active acid at a temperature of about 25 to about 37° C., or a pharmaceutically acceptable salt thereof;
(b) at least one hydrophilic polymer which is not instantly soluble in gastric fluids; and
(c) at least one alkalizer;
wherein the composition reduces evacuation from the stomach; and
   provides at least about 70% release of the active for a period of time between about 7 to about 12 hours following oral administration.

In another aspect of the present invention relates to a solid pharmaceutical composition for the controlled release of an active agent in the gastrointestinal tract, comprising:
(a) at least one weak acid active agent with a solubility of less than about 0.1 mg/ml in an aqueous solution at a pH of at most about the pKa of the active acid at a temperature of about 25 to about 37° C., or a pharmaceutically acceptable salt thereof;
(b) at least one hydrophilic polymer which is not instantly soluble in gastric fluids; and
(c) at least one alkalizer;
wherein the composition reduces evacuation from the stomach; and
   provides at least about 70% release of the active for a period of time between about 7 to about 12 hours following oral administration.

A second aspect of the present invention relates to a method for producing a tablet.

### BRIEF DESCRIPTION OF THE DRAWINGS

FIG. 1a shows comparative dissolution profiles of Examples 1 through 5 provided herein. FIG. 1b shows the influence of rate controlling polymers and alkalizing agents on the dissolution profiles of formulation.

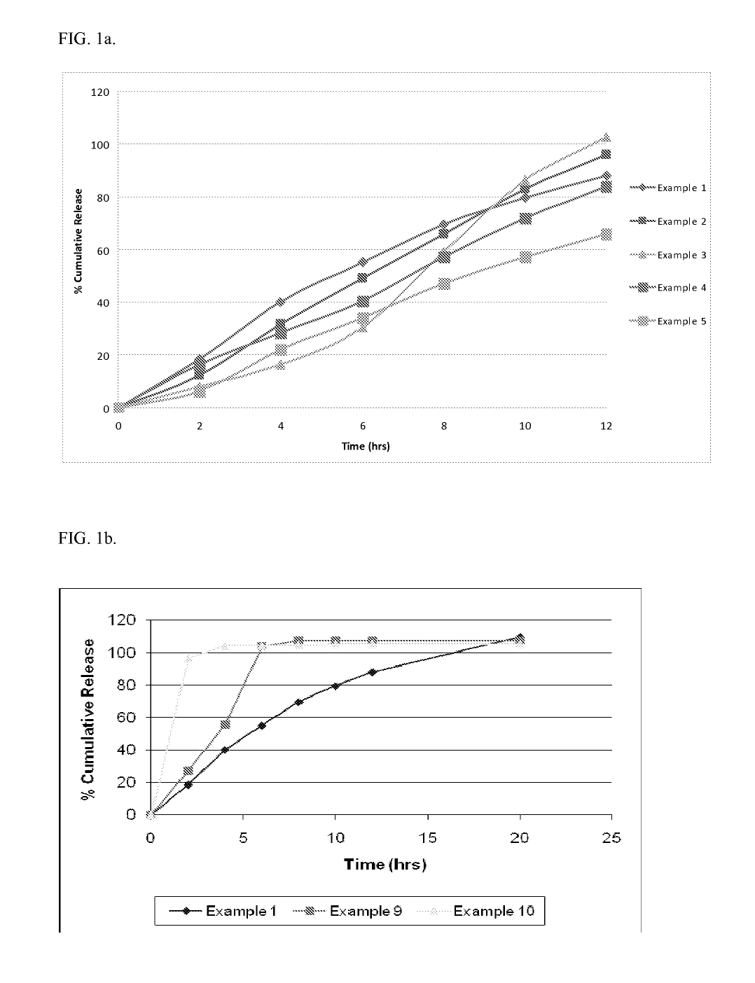

FIG. 2 shows the influence of the type and level of alkalizers and polymers on the dissolution rate of formulations

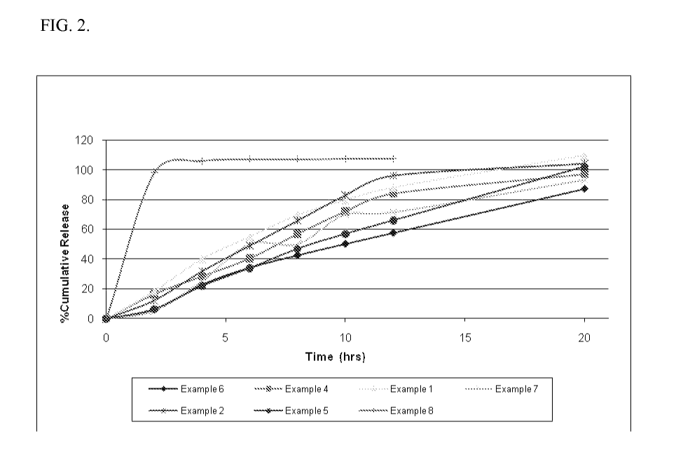

FIG. 3 shows the influence of pH of media on dissolution profiles. FIG. 3a shows the influence of pH on the dissolution of formulation containing Methocel K4M, magnesium oxide and calcium carbonate (Example 1) (acid robustness study). FIG. 3b shows the influence of pH on the dissolution of formulation containing Methocel K4M, Polyox WSR 1105 and sodium bicarbonate (Example 2) (acid robustness study).

FIG. 4 shows stability results. FIG. 4a shows the dissolution profiles of formulations containing Methocel K4M, magnesium oxide and calcium carbonate after storage at 40° C./75% RH for up to 3 months (Example 1). FIG. 4b shows dissolution profiles of formulations containing Methocel K4M, Polyox WSR 1105 and Sodium bicarbonate after storage at 40° C./75% RH for up to 3 months (Example 2).

FIG. 5 shows the influence of manufacturing process (direct compression vs. roller compaction) on the dissolution profile of Example 1 and Example 2 formulations.

### DETAILED DESCRIPTION OF THE INVENTION

### Definitions

As used herein, the following terms have the meanings ascribed to them unless specified otherwise.

The singular forms “a,” “an,” and, “the” include plural referents unless the context clearly dictates otherwise. Thus, for example, reference to a compound refers to one or more compounds or at least one compound. As such, the terms “a” (or “an”), “one or more”, and “at least one” can be used interchangeably herein.

The phrase “about” as used herein is used to provide flexibility to a numerical range endpoint by providing that a given value may be “a little above” or “a little below” the endpoint accounting for variations one might see in measurements taken among different instruments, samples, and sample preparations.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients.

The terms “therapeutic agent,” “active agent,” “bioactive agent,” “pharmaceutically active agent,” and “pharmaceutical,” and “drug” are used interchangeably herein to refer to a substance having a pharmaceutical, pharmacological, psychosomatic, or therapeutic effect. Further, when these terms are used, or when a particular active agent is specifically identified by name or category, it is understood that such recitation is intended to include the active agent per se, as well as pharmaceutically acceptable, pharmacologically active derivatives thereof, or compounds significantly related thereto, including without limitation, salts, pharmaceutically acceptable salts, N-oxides, prodrugs, active metabolites, isomers, fragments, analogs, solvates hydrates, radioisotopes, etc. Suitable agents for use in the present invention include, without limitation, compounds which have the formula (I):

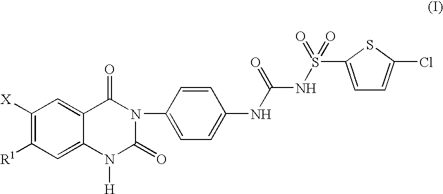

wherein:
R¹ is selected from the group consisting of H, halogen, —OH, —C₁₋₁₀-alkyl and C₁₋₆-alkylamino; and
X is selected from the group consisting of: F and I, or a pharmaceutically acceptable salt thereof; and combinations thereof. In a particularly preferred embodiment, the active agent is in a salt form such as that shown below, where the symbol M represents a suitable counterion.

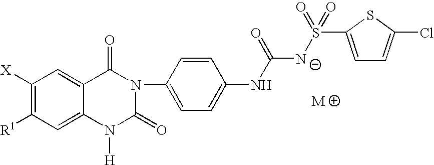

In a particularly preferred embodiment, the active agent is [4-(6-fluoro-7-methylamino-2,4-dioxo-1,4-dihydro-2H-quinazolin-3-yl)-phenyl]-5-chloro-thiophen-2-yl-sulfonylurea, in all suitable forms.

The present invention is applicable not only to [4-(6-fluoro-7-methylamino-2,4-dioxo-1,4-dihydro-2H-quinazolin-3-yl)-phenyl]-5-chloro-thiophen-2-yl-sulfonylurea, but also to other weakly acidic drugs with poor aqueous solubility. Examples of such drugs include, but are not limited to Indomethacin, Ketoprofen and Naproxen.

A “hydrophilic polymer” as used herein refers to a composition that comprises a polymer such as cellulose derivatives, dextrans, starches, carbohydrates, base polymers, natural or hydrophilic gums, xanthans, alginates, gelatins, polyacrylic acids, polyvinyl alcohol (PVA), polyvinyl pyrrolidone (PVP), carbomers, combinations thereof or the like.

As used herein, the terms “sustained release,” “prolonged release,” and “controlled release” as applied to drug formulations have the meanings ascribed to them in “Remington's Pharmaceutical Sciences,” 18.sup.th Ed., p. 1677, Mack Pub. Co., Easton, Pa. (1990). Sustained release drug systems include any drug delivery system which achieves the slow release of drug over an extended period of time, and include both prolonged and controlled release systems. If such a sustained release system is effective in maintaining substantially constant drug levels in the blood or target tissue, it is considered a controlled release drug delivery system. If, however, a drug delivery system extends the duration of action of a drug over that achieved by conventional delivery, without reference to whether it is successful at achieving substantially constant blood or tissue drug levels, it is considered a prolonged release system. The term “controlled release,” when used to describe the manner an active ingredient is released from a tablet, refers to the fact that the tablet is capable of releasing the active agent to the body for a prolonged period of time, e.g., for at least about 18 hours, and preferably for at least about 24 hours. Preferably, a controlled release tablet releases the active agent from the tablet gradually into the body. For example, a controlled release tablet that is designed to release of the active agent for about 7-12 hours preferably has the following dissolution specification using the dissolution test method described in the Examples: no more than 40% of the active agent (e.g., by weight) released in 1 hour, about 70-85% of the active agent released in 12 hours, and no less than about 80% of the active agent released at 24 hours. In another example, a sustained release tablet is designed to release the active agent at a nearly linear zero order rate (typically when the active agent dissolution is measured up to 70% of the active agent release).

Unless specified otherwise, a range of “molecular weight” of a polymer (e.g., a polyethylene oxide polymer or a polysaccharide) or a gelation facilitator agent (e.g., a polyethylene glycol) described below is a weighted average molecular weight (measured by gel permeation chromatography).

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment.

As used herein, the term “treating” refers to providing an appropriate dose of a therapeutic agent to a subject suffering from an ailment.

As used herein, the term “condition” refers to a disease state for which the compounds, compositions and methods of the present invention are being used against.

As used herein, the term “ADP-mediated disease or condition” and the like refers to a disease or condition characterized by less than or greater than normal, ADP activity. An ADP-mediated disease or condition is one in which modulation of ADP results in some effect on the underlying condition or disease (e.g., a ADP inhibitor or antagonist results in some improvement in patient well-being in at least some patients).

As used herein, “subject” refers to a mammal that may benefit from the administration of a drug composition or method of this invention. Examples of subjects include humans, and may also include other animals such as horses, pigs, cattle, dogs, cats, rabbits, rats, mice and aquatic mammals. In one specific aspect, a subject is a human.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “pharmaceutically acceptable carrier” and “carrier” may be used interchangeably, and refer to any inert and pharmaceutically acceptable material that has substantially no biological activity, and makes up a substantial part of the formulation.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

The term “dissolution” refers to the rate of the active agent dissolving in a liquid (medium) defined by the method. Suitable methods known in the art for determining the dissolution profile of a solid dosage form include, e.g., United States Pharmacopeia (USP) dissolution tests <711[KC1]> Apparatus 3.

The term “disintegration” refers to the disintegration of tablets or capsules when placed in a liquid medium in the experimental condition. Complete disintegration is defined as that state in which any residue of the unit, except fragments of insoluble coating or capsule shell, remaining on the screen of the test apparatus is a soft mass having no palpably firm core. Disintegration does not imply complete solution of the unit or even of its active constituent. Suitable methods known in the art for determining the disintegration time of a solid dosage form include, e.g., the USP disintegration test <701>. The term “non-disintegrating” refers to a composition that does not fully disintegrate in an hour or less in a suitable aqueous medium determined using the USP disintegration test. The term “slow-disintegrating” refers to a composition that fully disintegrates in about an hour to about 30 minutes in a suitable aqueous medium determined using the USP disintegration test.

The term “bioavailability” refers to the rate and/or extent to which a drug is absorbed or becomes available to the treatment site in the body.

As used herein, the terms “administration,” and “administering” refer to the manner in which an active agent is presented to a subject. Administration can be accomplished by various art-known routes such as oral, parenteral, transdermal, inhalation, implantation, etc.

The term “oral administration” represents any method of administration in which an active agent can be administered through the oral route by swallowing, chewing, or sucking an oral dosage form. Such solid or liquid oral dosage forms are traditionally intended to substantially release and or deliver the active agent in the gastrointestinal tract beyond the mouth and/or buccal cavity. Examples of solid dosage forms include conventional tablets, capsules, caplets, etc.

As used herein, “oral dosage form” refers to a formulation that is prepared for administration to a subject through the oral route of administration. Examples of known oral dosage forms, include without limitation, tablets, capsules, caplets, powders, pellets, granules, solutions, suspensions, solutions and solution pre-concentrates, emulsions and emulsion pre-concentrates, etc. In some aspects, powders, pellets, granules and tablets may be coated with a suitable polymer or a conventional coating material to achieve, for example, greater stability in the gastrointestinal tract, or to achieve the desired rate of release. Moreover, capsules containing a powder, pellets or granules may be further coated. Tablets may be scored to facilitate division of dosing. Alternatively, the dosage forms of the present invention may be unit dosage forms wherein the dosage form is intended to deliver one therapeutic dose per administration.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### DESCRIPTION OF THE EMBODIMENTS

In one aspect, the invention provides a solid pharmaceutical composition for the controlled release of an active agent in the gastrointestinal tract, comprising:

(a) at least one weak acid active agent with a solubility of less than about 0.3 mg/ml in an aqueous solution at a pH of at most about the pKa of the active acid at a temperature of about 37° C., or a pharmaceutically acceptable salt thereof;
(b) at least one hydrophilic polymer which is not instantly soluble in gastric fluids; and
(c) an alkalizer, wherein the composition reduces evacuation from the stomach; and
provides at least about 70% release of the active for a period of time from about between about 7 to about 12 hours following oral administration.

In another aspect, the invention provides a solid pharmaceutical composition for the controlled release of an active agent in the gastrointestinal tract, comprising:

(a) at least one weak acid active agent with a solubility of less than about 0.2 mg/ml in an aqueous solution at a pH of at most about the pKa of the active acid at a temperature of about 37° C., or a pharmaceutically acceptable salt thereof;
(b) at least one hydrophilic polymer which is not instantly soluble in gastric fluids; and
(c) an alkalizer, wherein the composition reduces evacuation from the stomach; and
provides at least about 70% release of the active for a period of time from about between about 7 to about 12 hours following oral administration.

In another aspect, the invention provides a solid pharmaceutical composition for the controlled release of an active agent in the gastrointestinal tract, comprising:

(a) at least one weak acid active agent with a solubility of less than about 0.1 mg/ml in an aqueous solution at a pH of at most about the pKa of the active acid at a temperature of about 37° C., or a pharmaceutically acceptable salt thereof;
(b) at least one hydrophilic polymer which is not instantly soluble in gastric fluids; and
(c) an alkalizer, wherein the composition reduces evacuation from the stomach; and
provides at least about 70% release of the active for a period of time from about between about 7 to about 12 hours following oral administration.

In one aspect, the formulation may float and is non-disintegrating upon hydration in gastric fluid. In another aspect the formulation may float and is slow-disintegrating upon hydration in gastric fluid. In another aspect the composition reduces evacuation from the stomach.

The composition, comprising at least one hydrophilic polymer and an alkalizer forms a matrix for the active agent in the composition. The composition provides a desired release profile for the active agent, specifically a controlled release of at least about 70% of the active from the tablet into the stomach for a period of time from about between about 7 to about 12 hours following oral administration. Depending on the ultimate use of the tablets, these tablets typically comprise components that are physiologically or pharmacologically acceptable.

In one aspect, the invention provides a solid composition wherein the tablet provides near zero order release profile independent of a pH range of about 1 to about 7.4.

The first polymer is water insoluble and contributes to forming a network of materials within the matrix which can swell upon absorbing water. The second polymer comprises at least one polymer, or it may comprise a mixture of two or more polymers. In one aspect, polysaccharides are the preferred type of polymer(s) in the second polymer. Also water insoluble, the second polymer interacts with the first polymer to form a matrix that is more resistant to erosion in the digestive tract and can further retard the release of the active agent from the tablet. The gelation facilitator agent is a hydrophilic base that draws water into the core of the gel-forming matrix of the tablet, thereby allowing a substantially complete gelation of the entire tablet before the tablet reaches the large intestine. Preferably, the gelation facilitator agent has a solubility higher than about 0.1 gram/ml in water at a temperature of about 37° C. Different forms and/or types of the polymers and the gelation facilitator agent can be used to modify the gelation rate and/or erosion rate of the gel matrix. They can be selected to provide a controlled release pattern of the active agent-containing particles. Other additives can be incorporated to further modify the gelation and/or release pattern of the active agent.

The particle is formulated to further modify the release of the active agent (in particular the hydrophilic agent) from the tablet. Typically, the particle comprises an active agent and an optional coating material on, and preferably around, the active agent. The active agent can be in any suitable form. In certain embodiments, the active agent can be in the form of an amorphous solid, a crystal, a granule, or a pellet. These active agent forms may facilitate certain coating processes of the active agents. Moreover, the particle can comprise a single active agent crystal (or granule or pellets or amorphous solid) or can comprise a plurality of active agent crystals (or granules or pellets or amorphous solid).

In another aspect, the tablets are designed to have pulsatile or delayed onset release profiles. This can be achieved by designing, e.g., a multilayered tablet or compression coated tablet. Different layers of the multilayered tablet can have different active agents, different amounts of active agents, different forms of active agents, different amounts or kinds of coating materials, different amounts or kinds of gel-forming materials, etc.

In a further aspect, the invention provides a method for generating a predetermined profile of sustained release of an active ingredient from a tablet of the present invention by choosing proper weight percentages of the first polymer, the second polymer, and the gelation facilitator agent in the gel-forming material. A maximal delaying effect in releasing an active agent can be achieved by including a coating material around the particle(s).

### Active Agents

In one set of embodiments, the active agents of the present invention are selected from the class of compounds in the dihydroquinazolinylphenyl thiophenyl sulfonylurea family and are useful in the treatment of conditions such as thrombosis. Illustrative examples of suitable dihydroquinazolinylphenyl thiophenyl sulfonylurea compounds for use in the present invention have the formula (I):

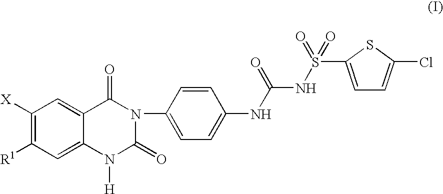

wherein:
R¹ is selected from the group consisting of H, halogen, —OH, —C₁₋₁₀-alkyl and C₁₋₆-alkylamino; and
X is selected from the group consisting of: F and I.

More preferably, the agent is [4-(6-fluoro-7-methylamino-2,4-dioxo-1,4-dihydro-2H-quinazolin-3-yl)-phenyl]-5-chloro-thiophen-2-yl-sulfonylurea, in all suitable forms. In one aspect, the invention provides a solid composition, wherein the active agent is [4-(6-fluoro-7-methylamino-2,4-dioxo-1,4-dihydro-2H-quinazolin-3-yl)-phenyl]-5-chloro-thiophen-2-yl-sulfonylurea potassium salt. Methods for the preparation of compounds of formula (I) are described in US-2007-0123547-A1.

Surprisingly, it was found that compounds of formula (I) are weak acids with poor aqueous solubility at acidic pH. Thus, in one embodiment, the active agents of the present invention are a poorly soluble weak acid compound in its salt form that has aqueous solubility of less than 0.1 mg/ml at pH 1.0-7.4 at a temperature of about 37° C. having an ionized form and an un-ionized form. The aqueous solubility increases at a higher pH (e.g. >1 mg/ml at pH 8 or above). In certain instances, the active agent is initially present at least partly in an ionized form. In certain other instances, the active agent is initially present in an un-ionized form. In one embodiment and as described in more detail below, the alkalizer of the compositions described herein helps to increase the solubility of the active as pH increases up to pH 10 in a hydrate polymer matrix to enhance the product release profile. In another embodiment, the alkalizer of the compositions described herein helps to maintain substantially all of the active agent in its dissolved ionized form in the formulation when it is hydrated in the stomach.

In another set of embodiments, the active agents of the present invention are selected from the class of NSAIDs and are useful in the treatment of conditions such as inflammation. Illustrative examples of suitable NSAIDs for use in the present invention include but are not limited to indomethacin, ketoprofen and naproxen.

In another set of embodiments, the active agents of the present invention are any weakly acidic drug, or a pharmaceutically acceptable salt thereof with poor aqueous solubility.

Any other suitable active agents can be included in the embodiments of the invention. For example, the pharmaceutically active agents include, but are not limited to, e.g., anti-inflammatory, antipyretic, anticonvulsant and/or analgesic agents such as indomethacin, diclofenac, diclofenac Na, ibuprofen, phenylbutazone, oxyphenbutazone, mepirizol, aspirin, ethenzamide, aminopyrine, phenacetin, scopolamine butylbromide, morphine, etomidoline, pentazocine, fenoprofen calcium, etc; tuberculostats such as isoniazid, ethambutol hydrochloride, etc.; cardiocirculatory system drugs such as isosorbide dinitrate, nitroglycerin, nifedipine, dipyridamole, arinone, methyldopa, furosemide, spironolactone, reserpine, neomapride, haloperidol, perphenazine, diazepam, lorazepam, chlordiazepoxide, etc.; antihistaminic agents such as chlorpheniramine maleate, etc.; vitamins such as thiamine nitrate, tocopherol acetate, cyclothiamine, pyridoxal phosphate, cobamamide, ascorbic acid, nicotinamide, etc.; antigout agents such as allopurinol, colchicine, probenecid, etc.; active sedatives such as amobarbital, bromovalerylurea, midazolam, chloral hydrate, etc.; antineoplastic agents such as fluorouracil, carmofur, cyclophosphamide, thiotepa, etc.; anticongestants such as phenylpropanolamine, etc.; antidiabetics such as acetohexamide, insulin, tolbutamide, etc.; diuretics such as hydrochlorothiazide, polythiazide, triamterene, etc.; bronchodilators such as aminophylline, theophylline, etc; antitussives such as, noscapine, dextromethorphan, etc; antiarrhythmic agents such as, procainamide, etc.; surface anesthetics such as ethyl aminobenzoate, lidocaine, etc.; antiepileptics such as phenyloin, ethosuximide, primidone, etc.; synthetic adrenocortical steroids such as hydrocortisone, prednisolone, triamcinolone, betamethasone, etc.; digestive system drugs such as famotidine, cimetidine, sucralfate, sulpiride, teprenone, plaunotol, etc.; central nervous system drugs such as indeloxazine, idebenone, calcium hopantenante, etc.; hyperlipemia treating agents such as pravastatin sodium etc.; and antibiotics such as cefotetan, josamycin and so on. Typical pharmaceutically active agents include, but are not limited to, e.g., anti-inflammatory, antipyretic, anticonvulsant and/or analgesic agents such as indomethacin, diclofenac, diclofenac Na, ibuprofen, aspirin, fenoprofen calcium, etc; cardiocirculatory system drugs such as methyldopa, furosemide, neomapride, etc.; vitamins such as ascorbic acid etc.; antigout agents such as probenecid, etc.; active sedatives such as amobarbital, etc.; antidiabetics such as acetohexamide, tolbutamide, etc.; diuretics such as hydrochlorothiazide, polythiazide, etc.; bronchodilators such as aminophylline, theophylline, etc; antiepileptics such as phenyloin, ethosuximide, primidone, etc.; digestive system drugs such as sulpiride etc.; central nervous system drugs such as calcium hopantenante, etc.; hyperlipemia treating agents such as pravastatin sodium etc.; and antibiotics such as cefotetan, josamycin and so on. Typical drugs among the above drugs are indomethacin, diazepamtheophylline, and the like.

As used herein, the term “active agent” includes all pharmaceutically acceptable forms of the active agent being described. For example, the active agent can be in a isomeric mixture, a solid complex bound to an ion exchange resin, or the like. In addition, the active agent can be in a solvated form. The term “active agent” is also intended to include all pharmaceutically acceptable salts, derivatives, and analogs of the active agent being described, as well as combinations thereof. For example, the pharmaceutically acceptable salts of the active agent include, without limitation, the sodium, potassium, calcium, magnesium, ammonium, tromethamine, L-lysine, L-arginine, N-ethylglucamine, N-methylglucamine and salt forms thereof, as well as combinations thereof and the like. Any form of the active agent is suitable for use in the compositions of the present invention, e.g., a pharmaceutically acceptable salt of the active agent, a free acid of the active agent, or a mixture thereof.

In yet another embodiment, an active agent is a drug that is unstable if it is in contact with simulated gastric fluid or a gel-forming matrix for a prolonged period of time at low pH (e.g., sensitive to low pH microenvironment)

In the embodiments of the invention, the active agent can be in any suitable form. For example, it can be in the form of a powder, pellet, or a granule (i.e., an aggregate of smaller units of active agent). An active agent can be pelletized or granulated using any suitable methods known in the art. Pelletization by extrusion (followed by spheronization) or granulation (wet or dry) is commonly defined as a size-enlargement process in which small particles are gathered into larger, aggregates in which the original particles can still be identified.

Any suitable granulation methods can be used to produce particles comprising an active agent. By definition, granulation is any process of size enlargement whereby small particles are gathered together into larger, aggregates to render them into a free-flowing state. For example, either wet granulation or dry granulation methods can be used.

Dry granulation refers to the granulation of a formulation without the use of heat and solvent. Dry granulation technology generally includes slugging or roller compaction. Slugging consists of dry-blending, compressing the formulation into a tablet (or slug) on a compression machine and milling to yield the granules. Roller compaction is similar to slugging, but a roller compactor is used instead of the tableting machines to form compact for milling. See, e.g., Handbook of Pharmaceutical Granulation Technology, D. M. Parikh, eds., Marcel-Dekker, Inc. pages 102-103 (1997). Dry granulation technique is useful in certain instances, e.g., when the active agent is sensitive to heat, water or solvent.

Alternatively, the active agents are granulated with high shear mixer granulation (“HSG”) or fluid-bed granulation (“FBG”). Both of these granulation processes provide enlarged granules but differ in the apparatuses used and the mechanism of the process operation. Blending and wet massing by HSG is accomplished by an impeller and a chopper in the mixer. Mixing, densification, and agglomeration of wetted materials are achieved through shearing and compaction forces exerted by the impeller. The wet mass is dried using commercial equipment such as a tray drier or a fluid-bed drier.

On the other hand, fluidization is the operation by which a mass of powder is manipulated to exhibit fluid-like characteristics using a gas or air as the fluidization vehicle. Such a fluidized bed resembles a vigorously boiling fluid, with solid particles undergoing turbulent motion, which can be generally increased with gas velocity. FBG is then a process by which granules are produced by spraying and drying a binder solution onto a fluidized powder bed to form larger granules in a fluidbed dryer. The binder solution can be sprayed from, e.g., one or more spray guns positioned at any suitable manner (e.g., top or bottom). The spray position and the rate of spray may depend on the nature of the active agent and the binder(s) used, and are readily determinable by those skilled in the art.

Optionally, granulated active agents can be milled after wet granulation or drying. Milling can be performed using any commercially available equipment, e.g., COMIL® equipped with a 0.039 inch screen. The mesh size for the screen of a COMIL® can be selected depending on the size of the active agent granule or pellet desired. Typically, the mesh size can range from 0.331 inch screen (mesh 20) to 0.006 inch screen (mesh 100). The milling process aids in providing relatively uniform granule size. After the wet granulated active agents are milled, they may be further dried (e.g., in a fluidbed drier) if desired.

Typically, the mean size of the active granule can range from about 20 μm to about 3 mm, optionally about 50 μm to about 2 mm, about 100 μm to about 1 mm. Typically, the bulk density or the tap density of the active agent granules range from about 0.1 g/ml to about 1.5 g/ml, optionally about 0.3 to about 0.8 g/ml, optionally about 0.4 g/ml to about 0.6 g/ml. Bulk density is measured based on USP method (see US testing method <616>).

### Hydrophilic Polymers

Surprisingly, it was found that the dissolution rate and absorption could be optimized by the combination of at least one hydrophilic polymer and at least one alkalizer. Not all hydrophilic water-soluble polymer conventional in the pharmaceutical arts may be used. Examples of hydrophilic polymers suitable for use in the present invention include, but are not limited to, cellulose derivatives, cellulose ether, polyethylene oxide, dextrans, starches, carbohydrates, base polymers, natural or hydrophilic gums, xanthans, pectin, alginates, mucin, agar, gelatins, polyacrylic acids, polyvinyl alcohol (PVA), polyvinyl pyrrolidone (PVP), carbomers, natural gum or the like. The hydrophilic polymers can be used individually, as well as in mixtures of two or several hydrophilic polymers. In the case of the cellulose derivatives, the alkyl or hydroxyalkyl cellulose derivatives, the alkyl or hydroxyalkyl cellulose derivatives preferably come into consideration such as example, methyl cellulose, ethylcellulose (EC), hydroxy methylcellulose, hydroxyethyl cellulose (HEC), hydroxypropyl cellulose (HPC), hydroxypropyl methylcellulose (HPMC), methylhydroxy ethylcellulose, methylhydroxy ethylcellulose, methylhydroxy propylcellulose or sodium carboxymethyl cellulose. Suitable cellulose based hydrophilic polymers may have various degrees of substitution and/or different molecular weights corresponding to a different degree of viscosity of the aqueous solution. In an embodiment of the present invention, the release rate controlling polymer may be selected from the group consisting of hydroxypropylmethyl cellulose (HPMC), hydroxyethyl cellulose, ethylcellulose, carbomer and combinations thereof.

The hydroxypropyl methylcellulose (HPMC) used as the release rate controlling polymer in the present invention may suitably be any HPMC conventional in the pharmaceutical art. The HPMC used may suitably be, for example, HPMC substitution types 1828, 2208, 2906 and 2910 as described in the USP. The hydroxypropyl methylcellulose used may suitably be, for example, METHOCEL™ as supplied by Dow Chemical Company. Similar HPMCs are also available from other suppliers. Preferably, the HPMC used is HPMC 2208, more preferably METHOCEL™ K4M Premium CR; METHOCEL™ K100M; hydroxypropylmethylcellulose (HPMC) [e.g., Metolose 90SH10000 (viscosity: 4100-5600 cps., 1% in H₂O, 20° C.), Metolose 90SH₅₀₀₀₀ (viscosity: 2900-3900 cps, under the same condition above), Metolose 90SH30000 (viscosity: 25000-35000 cps, 2% in H₂O, 20° C.), all of which are trade names of Shin-Etsu Chemicals Co.]. Additional suitable cellulosic polymers are sodium carboxymethylcellulose (CMC-Na) [e.g., Sanlose F-150MC (average mol. wt.: 2×10⁵, viscosity: 1200-1800 cps, 1% in H₂O, 25° C.), Sanlose F-1000MC (average mol. wt.: 42×10⁴; viscosity: 8000-12000 cps, under the same condition above), Sanlose F-300MC (average mol. wt.: 3×10⁵; viscosity: 2500-3000 cps, under the same condition above), all of which are trade names of Nippon Seishi Co., Ltd.]; hydroxyethylcellulose (HEC) [e.g., HEC Daicel SE850 (average mol. wt.: 148×10⁴; viscosity: 2400-3000 cps, 1% in H₂O, 25° C.), HEC Daicel SE900 (average mol. wt.: 156×10⁴; viscosity: 4000-5000 cps, under the same condition above), all of which are trade names of Daicel Chemical Industries]; carbonxyvinyl polymers [e.g., Carbopol 940 (average mol. wt.: ca. 25×10⁵; B.F. Goodrich Chemical Co.) and so on.

Polyox (Dow Chemical) which can be used in the present invention is a water-soluble polymer, polyethylene oxide, and has different viscosities and hydrophilicities in an aqueous solution depending on its average molecular weight. Suitable to serve as the hydrophilic polymers are polyethylene oxide polymers, e.g., POLYOX™ WRS-303 (average mol. wt.: 7×10⁶; viscosity: 7500-10000 cps, 1% in H₂O, 25° C.), POLYOX™ WSR Coagulant (average mol. wt.: 5×10⁶; viscosity: 5500-7500 cps, under the same condition above), POLYOX™ WSR-301 (average mol. wt.: 4×10⁶ viscosity: 1650-5500 cps, under the same condition above), POLYOX™ WSR-1105 (average mol. wt.: 900,000, 8800-17,600 viscosity: 1650-5500 cps, (5% solution) under the same condition above), POLYOX™ x WSR-N-60K (average mol. wt.: 2×10⁶; viscosity: 2000-4000 cps, 2% in H₂O, 25° C.).

Preferably, the composition includes POLYOX™ (polyethylene oxide, Dow Chemical) WSR 1105, cellulose ethers, e.g. Metolose (hydroxypropyl methylcellulose (HPMC), ShinEtsu), and/or their mixtures. These polymers are hydrated thereby increasing the viscosity and giving them their hydrophilic properties.

Carbopol (BFGoodrich) is an ionizable and hydrophilic polymer wherein an acrylic acid polymer is chemically cross-linked with polyalkenyl alcohol and divinyl glycol, and Carbopol 934P NF, 974P NF, 971P NF, etc. are used for oral use. Theses hydrophilic polymers form highly viscous gel and are swelled upon contacting with water.

In one aspect, the invention provides a solid composition wherein the amount of hydrophilic polymer is less than about 27.8% w/w of the composition. In one aspect, the invention provides a solid composition wherein the amount of hydrophilic polymer is between about 27.8% w/w to about 10 w/w % of the total composition. In one aspect, the invention provides a solid composition wherein the hydrophilic polymer has an average molecular weight of between about 0.82 and about 9×10⁵ Daltons. In one aspect the hydrophilic polymer has a viscosity of 8800 to 17,600 cps. In one aspect, the invention provides a solid composition wherein the at least one hydrophilic polymer is a combination of hydrophilic polymers. In one aspect, the invention provides a solid composition wherein the hydrophilic polymer is selected from the group consisting of a methocel cellulose ether, polyethylene oxide (PEO), and combinations thereof. In one aspect, the invention provides a solid composition wherein the methocel cellulose ether is METHOCEL™ K4M. In one aspect, the invention provides a solid composition wherein the polyethylene oxide is POLYOX™ WSR 1105. In one aspect, the invention provides a solid composition wherein the weight ratio of said METHOCEL™ K4M to said POLYOX™ WSR 1105 is from about 0.9 to about 0.69.

### Alkalizers

Formulations were designed to provide an alkaline micro-environment for these compounds along with controlled release hydrophilic polymers. The alkalizer is used to create a microenvironment in the formulation to optimize drug release after the polymer matrix is hydrated.

The alkalizers of the compositions described herein are capable of raising the pH of the micro-environment for these compounds in the hydrated formulation to a pH greater than about the pKa of the active acid, irrespective of the starting pH of stomach. In one embodiment, the alkalizers of the compositions described herein are capable of raising the pH of the micro-environment in the hydrated formulation to typically about 9.0-9.5, irrespective of the starting pH of stomach. In this way, the alkalizer helps increase the solubility of the active as pH increases up to pH 10 in a hydrate polymer matrix to enhance the product release/dissolution profile from the hydrated formulation. Although pH adjusting agents may be used with the alkalizers of the present invention, one skilled in the art will appreciate that acidic agents can also be used to adjust the pH of the alkalizer as long as the alkalizer as a whole raises the pH of the micro-environment for these compounds in the hydrated formulation to greater than about the pKa of the active acid.

Suitable alkalizer agents include, but are not limited to, organic and inorganic basic compounds of a wide range of aqueous solubilities and molecular weights and the like and mixtures thereof. Representative examples of inorganic basic salts include ammonium hydroxide, alkali metal salts, alkaline earth metal salts such as magnesium oxide, magnesium hydroxide, calcium hydroxide, sodium hydroxide, potassium hydroxide, aluminum hydroxide, potassium carbonate, sodium bicarbonate and the like and mixtures thereof. In one aspect, the invention provides a solid composition wherein the alkalizer selected from the group consisting of calcium carbonate, magnesium oxide, sodium bicarbonate and arginine and pharmaceutically acceptable salts thereof. The solubility and the molecular size of the alkalizer may affect its diffusion rate in the hydrated product matrix and influence the dissolution profile of the active agent.

In one aspect, the invention provides a solid composition wherein amount of alkalizer is from about 5 to about 50 weight percent of the total composition. In one aspect, the invention provides a solid composition wherein the combined weight percent of the alkalizer is greater than or equal to the weight percent of the active. In one aspect, the invention provides a solid composition wherein the weight ratio of said alkalizer to said hydrophilc polymer is from about 0.9 to about 0.69. In one aspect, the invention provides a solid composition wherein said composition comprises from about 7.6% w/w to about 8.9% w/w active; from about 27.8% w/w to about 15% w/w hydrophilic polymer; and from about 15% w/w to about 30% w/w alkalizer of the total composition.

In one aspect, the invention provides a solid composition of claim 1, wherein the composition provides at least about 70% release of the active between about 10 to about 12 hours following oral administration.

In one embodiment, the present invention provides binary alkalizers for example comprising a carbonate salt and a second alkalizer, magnesium oxide. The concentration of each alkalizer component is tailored such that the final pH of the micro-environment for these compounds is achieved and sustained for a period of time, e.g., for at least about 7 hours, at least about 8 hours, at least about 9 hours, at least about 10 hours, at least about 11 hours, or at least about 12 hours. This typically involves a trial and error type of procedure of adding various amounts of each alkalizer component and then measuring the final pH over time. In this way, selection of an appropriate weight ratio for each alkalizer component can be easily determined in just a few trials. For example, the weight ratio of carbonate salt to bicarbonate salt can be from about 1:10 to about 10:1, preferably from about 1:5 to about 5:1, more preferably from about 1:3 to about 3:1, and still more preferably from about 1:2 to about 2:1.

The carbonate salt is generally selected from sodium carbonate, potassium carbonate, calcium carbonate, ammonium carbonate, and magnesium carbonate. Preferably, the carbonate salt is calcium carbonate. Similarly, the bicarbonate salt is generally selected from sodium bicarbonate, potassium bicarbonate, calcium bicarbonate, and magnesium bicarbonate. Preferably, the bicarbonate salt is sodium bicarbonate or potassium bicarbonate. Most preferably, the bicarbonate salt is sodium bicarbonate. In some embodiments, sodium bicarbonate is preferred. The amount of carbonate salt and bicarbonate salt used in the binary alkalizer is an amount that is sufficient to raise pH of the micro-environment for these compounds in the hydrated formulation to a pH of about the pKa of the active acid or more, preferably about 8.5 or more, and more preferably about 9 or more (e.g., about 9-11), irrespective of the starting pH.

In certain instances when binary alkalizers are used, the amount of bicarbonate salt is greater than or equal to the amount of carbonate salt, and the weight ratio of carbonate salt to bicarbonate salt is from about 1:1 to about 1:10, preferably from about 1:1 to about 1:5, and more preferably from about 1:1 to about 1:2, e.g., 1:1, 1:1.1, 1:1.2, 1:1.3, 1:1.4, 1:1.5, 1:1.6, 1:1.7, 1:1.8, 1:1.9, or 1:2. Alternatively, the amount of bicarbonate salt is less than or equal to the amount of carbonate salt, and the weight ratio of carbonate salt to bicarbonate salt is from about 1:1 to about 10:1, preferably from about 1:1 to about 5:1, and more preferably from about 1:1 to about 2:1, e.g., 1:1, 1.1:1, 1.2:1, 1.3:1, 1.4:1, 1.5:1, 1.6:1, 1.7:1, 1.8:1, 1.9:1, or 2:1. In certain other instances, the combined amount of carbonate salt and bicarbonate salt is greater than or equal to the amount of the active agent, and the weight ratio of carbonate salt and bicarbonate salt to active agent is preferably from about 1:1 to about 10:1, e.g., 1:1, 2:1, 3:1, 4:1, 5:1, 6:1, 7:1, 8:1, 9:1, or 10:1. Alternatively, the combined amount of carbonate salt and bicarbonate salt is less than or equal to the amount of the active agent, and the weight ratio of carbonate salt and bicarbonate salt to active agent is preferably from about 1:1 to about 1:10, e.g., 1:1, 1:2, 1:3, 1:4, 1:5, 1:6, 1:7, 1:8, 1:9, or 1:10.

In view of the above, the alkalizers of the present invention, in some embodiments, are binary alkalizers containing sodium carbonate and sodium bicarbonate.

Alternatively, in another embodiment, the alkalizers of the present invention are binary alkalizers, for example comprising a carbonate salt or a bicarbonate salt and a second alkalizer, for example magnesium oxide. The concentration of each alkalizer component is tailored such that the final pH of the micro-environment for these compounds in the hydrated formulation is achieved and sustained for a period of time, e.g., for at least about 7 hours, at least about 8 hours, at least about 9 hours, at least about 10 hours, at least about 11 hours, or at least about 12 hours. Selection of an appropriate weight ratio for each alkalizer component can be easily determined to achieve the dissolution profile in gastric fluid. For example, the weight ratio of carbonate salt to bicarbonate salt can be from about 1:10 to about 10:1, preferably from about 1:5 to about 5:1, more preferably from about 1:3 to about 3:1, and still more preferably from about 1:2 to about 2:1

Suitable carbonate salts and bicarbonate salts are described above. The amount of carbonate salt or bicarbonate salt used in the binary alkalizer is an amount that is sufficient, when used with the second alkalizer to raise pH of the micro-environment for these compounds in the hydrated formulation to a pH of about the pKa of the active acid or more, preferably about 8.5 or more, and more preferably about 9 or more (e.g., about 9-11), irrespective of the starting pH. In certain instances, the amount of the second alkalizer in the binary alkalizer is greater than or equal to the amount of the carbonate salt or bicarbonate salt. For example, the weight ratio of the second alkalizer to the carbonate salt or bicarbonate salt can be from about 1:1 to about 10:1, preferably from about 1:1 to about 5:1, and more preferably from about 1:1 to about 3:1. In certain other instances, the amount of the second alkalizer in the binary alkalizer is less than or equal to the amount of the carbonate salt or bicarbonate salt. For example, the weight ratio of the second alkalizer to the carbonate salt or bicarbonate salt can be from about 1:1 to about 1:10, preferably from about 1:1 to about 1:5, and more preferably from about 1:1 to about 1:3.

The second alkalizer is generally selected from a metal oxide such as magnesium oxide or aluminum oxide; a phosphate salt such as monobasic sodium phosphate, dibasic sodium phosphate, monobasic potassium phosphate, dibasic potassium phosphate, monobasic calcium phosphate, dibasic calcium phosphate, monobasic magnesium phosphate, dibasic magnesium phosphate, monobasic ammonium phosphate, and dibasic ammonium phosphate. However, one skilled in the art will appreciate that any metal oxide or salt of citric acid, phosphoric acid, boric acid, ascorbic acid, or acetic acid is suitable for use in the alkalizers of the present invention. The amount of the second alkalizer used in the binary alkalizer is an amount that is sufficient, when used with the carbonate salt or bicarbonate salt, to raise pH of the micro-environment for these compounds in the hydrated formulation to a pH of about the pKa of the active acid or more. Typically this is about 9.0 to about 9.5 irrespective of the starting pH. Preferably about 8.5 or more, and more preferably about 9 or more (e.g., about 9-11), irrespective of the starting pH. In some embodiments, a metal oxide such as magnesium oxide or aluminum oxide is the preferred second alkalizer. In a particularly preferred embodiment, the metal oxide is amorphous magnesium oxide.

Alternatively, in yet another embodiment, the alkalizers of the present invention are binary alkalizers comprising a metal oxide and a citrate, phosphate, or borate salt. The concentration of each alkalizer component is tailored such that the final pH is achieved and sustained for a period of time, e.g., for at least about 7 hours, at least about 8 hours, at least about 9 hours, at least about 10 hours, at least about 11 hours, or at least about 12 hours.

Suitable metal oxides include, without limitation, magnesium oxide and aluminum oxide. Suitable citrate, phosphate, and borate salts include, without limitation, essentially any salt of citric acid, phosphoric acid, or boric acid known in the art such as those described above. In certain instances, the binary alkalizer comprises a metal oxide and a citrate salt. In certain other instances, the binary alkalizer comprises a metal oxide and a phosphate salt. In further instances, the binary alkalizer comprises a metal oxide and a borate salt. The amount of the metal oxide used in the binary alkalizer is an amount that is sufficient, when used with the citrate, phosphate, or borate salt, to raise pH of the micro-environment for these compounds in the hydrated formulation to a pH of about the pKa of the active acid or more, preferably about 8.5 or more, and more preferably about 9 or more (e.g., about 9-11), irrespective of the starting pH. Similarly, the amount of the citrate, phosphate, or borate salt used in the binary alkalizer is an amount that is sufficient, when used with the metal oxide, to raise pH of the micro-environment for these compounds in the hydrated formulation to a pH of about the pKa of the active acid or more, preferably about 8.5 or more, and more preferably about 9 or more (e.g., about 9-11), irrespective of the starting pH.

In certain instances, the amount of the metal oxide in the binary alkalizer is greater than or equal to the amount of the citrate, phosphate, or borate salt. For example, the weight ratio of the metal oxide to the citrate, phosphate, or borate salt can be from about 1:1 to about 10:1, preferably from about 1:1 to about 5:1, and more preferably from about 1:1 to about 3:1. In certain other instances, the amount of the metal oxide in the binary alkalizer is less than or equal to the amount of the citrate, phosphate, or borate salt. For example, the weight ratio of the metal oxide to the citrate, phosphate, or borate salt can be from about 1:1 to about 1:10, preferably from about 1:1 to about 1:5, and more preferably from about 1:1 to about 1:3.

Alternatively, in still yet another embodiment, the alkalizers of the present invention are ternary alkalizers comprising a carbonate salt, a bicarbonate salt, and a third alkalizer. The concentration of each alkalizer component is tailored such that the final pH of the micro-environment for these compounds is achieved and sustained for a period of time, e.g., for at least about 7 hours, at least about 8 hours, at least about 9 hours, at least about 10 hours, at least about 11 hours, or at least about 12 hours. The procedure described above for determining an appropriate weight ratio for each alkalizer component can also be applied to ternary alkalizers.

Suitable carbonate salts and bicarbonate salts are described above. The amount of carbonate salt and bicarbonate salt used in the ternary alkalizer is an amount that is sufficient, when used with the third alkalizer, to raise pH of the micro-environment for these compounds in the hydrated formulation to a pH of about the pKa of the active acid or more, preferably about 8.5 or more, and more preferably about 9 or more (e.g., about 9-11), irrespective of the starting pH.

The third alkalizer is generally selected from a metal oxide, a citrate salt, a phosphate salt, a borate salt, an ascorbate salt such as potassium ascorbate or sodium ascorbate, an acetate salt such as potassium acetate or sodium acetate, and alkaline starch. Suitable metal oxides include, without limitation, magnesium oxide and aluminum oxide. Suitable citrate, phosphate, and borate salts include, without limitation, any salt of citric acid, phosphoric acid, or boric acid known in the art such as those described above. The amount of the third alkalizer used in the ternary alkalizer is an amount that is sufficient, when used with the remaining components, to raise pH of the micro-environment for these compounds in the hydrated formulation to a pH of about the pKa of the active acid or more, preferably about 8.5 or more, and more preferably about 9 or more (e.g., about 9-11), irrespective of the starting pH. In some embodiments, a metal oxide such as magnesium oxide or aluminum oxide is the preferred third alkalizer. In a particularly preferred embodiment, the metal oxide is amorphous magnesium oxide.

In certain instances, the amount of the carbonate salt or bicarbonate salt in the ternary alkalizer is greater than or equal to the amount of the third alkalizer. For example, the weight ratio of the carbonate salt or bicarbonate salt to the third alkalizer can be from about 1:1 to about 10:1, preferably from about 1:1 to about 5:1, and more preferably from about 1:1 to about 3:1. In certain other instances, the amount of the carbonate salt or bicarbonate salt in the ternary alkalizer is less than or equal to the amount of the third alkalizer. For example, the weight ratio of the carbonate salt or bicarbonate salt to the third alkalizer can be from about 1:1 to about 1:10, preferably from about 1:1 to about 1:5, and more preferably from about 1:1 to about 1:3.

The ternary alkalizers of the present invention, in some of the most preferred embodiments, contain sodium carbonate, sodium bicarbonate, and amorphous magnesium oxide. In certain instances, the amount of sodium bicarbonate is greater than or equal to the amount of sodium carbonate. For example, the weight ratio of sodium bicarbonate to sodium carbonate can be from about 1:1 to about 10:1, preferably from about 1:1 to about 5:1, and more preferably from about 1:1 to about 3:1. In certain other instances, the amount of amorphous magnesium oxide is greater than or equal to the combined amount of sodium carbonate and sodium bicarbonate. For example, the weight ratio of amorphous magnesium oxide to sodium carbonate and sodium bicarbonate can be from about 1:1 to about 10:1, preferably from about 1:1 to about 5:1, and more preferably from about 1:1 to about 3:1.

Alternatively, in a further embodiment, the alkalizers of the present invention are alkalizers comprising a carbonate salt or a bicarbonate salt and one or more alkalizers selected from the group consisting of a metal oxide. The concentration of each alkalizer component is tailored such that the final pH of the micro-environment for these compounds in the stomach is achieved and sustained for a period of time, e.g., for at least about 7 hours, at least about 8 hours, at least about 9 hours, at least about 10 hours, at least about 11 hours, or at least about 12 hours.

Suitable carbonate salts and bicarbonate salts are described above. The amount of carbonate salt or bicarbonate salt used in the alkalizer is an amount that is sufficient, when used with the remaining components, to raise pH of the micro-environment for these compounds in the hydrated formulation to a pH of about the pKa of the active acid or more, preferably about 8.5 or more, and more preferably about 9 or more (e.g., about 9-11), irrespective of the starting pH.

The one or more alkalizers are generally selected from a metal oxide, a carbonate salt, and a bicarbonate salt. Suitable metal oxides include, without limitation, magnesium oxide and aluminum oxide. The amount of the additional alkalizer s used in the alkalizer is an amount that is sufficient, when used with the carbonate salt or bicarbonate salt, to raise pH of the micro-environment for these compounds in the hydrated formulation to a pH of about the pKa of the active acid or more, preferably about 8.5 or more, and more preferably about 9 or more (e.g., about 9-11), irrespective of the starting pH.

In certain instances, the alkalizer comprises a carbonate salt or a bicarbonate salt, a metal oxide, and a citrate, phosphate, or borate salt. In certain other instances, the alkalizer comprises a carbonate salt or a bicarbonate salt, a citrate salt, and a phosphate salt. In certain instances, the alkalizer comprises a carbonate salt or a bicarbonate salt, a citrate salt, and a borate salt. In certain other instances, the alkalizer comprises a carbonate salt or a bicarbonate salt, a phosphate salt, and a borate salt. Preferably, the metal oxide is amorphous magnesium oxide.

In certain instances, the amount of the carbonate salt or bicarbonate salt in the alkalizer is greater than or equal to the amount of the metal oxide or the citrate, phosphate, or borate salt. For example, the weight ratio of the carbonate salt or bicarbonate salt to the metal oxide or the citrate, phosphate, or borate salt can be from about 1:1 to about 10:1, preferably from about 1:1 to about 5:1, and more preferably from about 1:1 to about 3:1. In certain other instances, the amount of the carbonate salt or bicarbonate salt in the alkalizer is less than or equal to the amount of the metal oxide or the citrate, phosphate, or borate salt. For example, the weight ratio of the carbonate salt or bicarbonate salt to the metal oxide or the citrate, phosphate, or borate salt can be from about 1:1 to about 1:10, preferably from about 1:1 to about 1:5, and more preferably from about 1:1 to about 1:3.

While the foregoing discussion has focused on the ability of the alkalizer to alter pH to of the micro-environment for these compounds in the hydrated formulation increase the solubility of the active as pH increases up to pH 10 in a hydrate polymer matrix to enhance the product release profile, it is conceivable that the alkalizer may also have subsidiary beneficial effects on the extent of absorption in the stomach and the rest of the GI tract. For example, the alkalizer may create a pH of the micro-environment for these compounds in the hydrated formulation to regulate the release of the active agent gradually in the stomach without precipitation. This allows the active agent released to become un-ionized in the stomach acid for absorption. In addition, the alkalizer may create a pH of the micro-environment in the formulation to regulate the release of the drug to avoid a dramatic increase of concentration of the unionized drug agent at a lower pH in the stomach causing formation large aggregates to reduce the bioavailability. The use of a combination of polymer and alkalizer also allows for the control of disintegration, floatation properties and the mechanical strength of the hydrated formulation to achieve the gastric retentive properties. In one aspect, the invention provides a solid composition, wherein the composition consists of at least one alkalizer of carbonate salt or bicarbonate salt in the invention provides non-disintegrating formulation with floatation property when hydrated in stimulated gastric fluid (0.1N HCl). In another aspect, the invention provides a solid composition, wherein the composition consists of at least one alkalizer of carbonate salt or bicarbonate salt in the invention provides a slow-disintegrating formulation with floatation property when hydrated in stimulated gastric fluid (0.1N HCl). This is resulted from the liberation of carbon dioxide, a decrease of density and a sufficiently (mechanically) strong non-disintegrating or slow-disintegrating composition upon hydration. A non- or slow-disintegrating composition with floatation property may provide gastric retentive behavior upon oral administration for improvements of bioavailability and reducing dosing intervals. It is to be understood that these subsidiary beneficial effects of the alkalizer are within the general scope of the alkalizer and compositions herein described.

### Other Components and Dosage Forms

The compositions of the present invention may take the form of a non- or slow-disintegrating controlled release matrix tablets, pills, capsules, or the like. Preferably, the dosage form is a slow-disintegrating tablet.

While each subject or patient possesses unique factors that may affect the rate and extent of absorption of the therapeutic agents described herein, dosage forms such as dissolving tablets, containing hydrophilic polymer and an alkalizer described herein offer advantages over other traditional formulations for oral administration. For example, each of these dosage forms releases 70% of the active for a period of time from about between about 7 to about 12 hours following oral administration. Similarly, the bioavailability of the therapeutic agent is increased, thereby reducing the time to onset of therapeutic activity as compared to traditional dosage forms for oral administration.

In addition, the preferred dosage forms of the present invention (e.g., dissolving tablets) containing a hydrophilic polymer and an alkalizer described herein offer advantages over dosage forms for oral administration that do not contain the hydrophilic polymer and an alkalizer. Importantly, because the combination of the hydrophilic polymer and the alkalizer in the dosage forms of the present invention helps maintain the therapeutic agent in its ionized form and increase the solubility of the active as pH increases up to pH 10 in a hydrate polymer matrix to enhance the product release profile in a controlled manner. The bioavailability of the therapeutic agent is increased, and the time to onset of therapeutic activity is modulated as compared to dosage forms for oral administration that do not contain the hydrophilic polymer and an alkalizer.

As used herein, the term “dosage form” refers to physically discrete units suitable as unitary dosages for human subjects and other mammals, each unit containing a predetermined quantity of therapeutic agent calculated to produce the desired onset, tolerability, and therapeutic effects, in association with one or more suitable pharmaceutical excipients such as carriers. Methods for preparing such dosage forms are known or will be apparent to those skilled in the art. In other embodiments, a tablet dosage form of the present invention can be prepared according to the procedures set forth, for example, in Remington: The Science and Practice of Pharmacy, 20^{th} Ed., Lippincott, Williams & Wilkins (2003); Pharmaceutical Dosage Forms, Volume 1: Tablets, 2nd Ed., Marcel Dekker, Inc., New York, N.Y. (1989); and similar publications. The dosage form to be administered will, in any event, contain a quantity of the therapeutic agent in a therapeutically effective amount for relief of the condition being treated when administered in accordance with the teachings of this invention.

The compositions of the present invention comprise a active agent or a pharmaceutically acceptable salt thereof, a hydrophilic polymer and an alkalizer. Typically, the tablet compositions of the present invention comprise from about 0.001% to about 85.0% by weight of the active agent (in whatever chosen form, measured as per its free acid form), and more typically from about 1.0% to about 50.0%. In some embodiments, about 4.0% by weight of the active agent is used. One skilled in the art understands that the foregoing percentages will vary depending upon the particular source of active agent utilized, the amount of active agent desired in the final formulation, as well as on the particular release rate of active agent desired. The binary or ternary alkalizer(s) of the tablet composition provides for a final pH of the micro-environment for these compounds in the hydrated formulation in excess of at least about the pKa of the active acid, preferably at least about 8.5, and more preferably at least about 9 (e.g., about 9-11).

The compositions of the present invention can additionally include pH adjusting agents; antioxidants, such as butylated hydroxytoluene and butylated hydroxyanisole; plasticizers; glidants; protecting agents; elastiomeric solvents; bulking agents; wetting agents; emulsifying agents; solubilizing agents; lubricants; suspending agents; preserving agents such as methyl-, ethyl-, and propyl-hydroxy-benzoates; sweetening agents; flavoring agents; coloring agents; and disintegrating agents such as crospovidone as well as croscarmellose sodium and other cross-linked cellulose polymers.

As used herein, the term “carrier” refers to a typically inert substance used as a “diluent” or vehicle for a drug such as a therapeutic agent. The term also encompasses a typically inert substance that imparts cohesive qualities to the composition. Suitable carriers for use in the compositions of the present invention include, without limitation, a binder, a gum base, and combinations thereof. Non-limiting examples of binders include mannitol, sorbitol, xylitol, maltodextrin, lactose, dextrose, sucrose, glucose, inositol, powdered sugar, molasses, starch, cellulose, microcrystalline cellulose, polyvinylpyrrolidone, acacia gum, guar gum, tragacanth gum, alginate, extract of Irish moss, panwar gum, ghatti gum, mucilage of isapol husks, Veegum®, larch arabogalactan, gelatin, methylcellulose, ethylcellulose, carboxymethylcellulose, hydroxypropylmethylcellulose, polyacrylic acid (e.g., Carbopol), calcium silicate, calcium phosphate, dicalcium phosphate, calcium sulfate, kaolin, sodium chloride, polyethylene glycol, and combinations thereof. These binders can be pre-processed to improve their flowability and taste by methods known in the art such as freeze drying (see, e.g., Fundamentals of Freeze-Drying, Pharm. Biotechnol., 14:281-360 (2002); Lyophililization of Unit Dose Pharmaceutical Dosage Forms, Drug. Dev. Ind. Pharm., 29:595-602 (2003)); solid-solution preparation (see, e.g., U.S. Pat. No. 6,264,987); and lubricant dusting and wet-granulation preparation with a suitable lubricating agent (see, e.g., Remington: The Science and Practice of Pharmacy, supra). For example, Mannogem® and Sorbogem®, sold by SPI Pharma Group (New Castle, Del.), are freeze-dried processed forms of mannitol and sorbitol, respectively. Typically, the compositions of the present invention comprise from about 25% to about 90% by weight of the binder, and preferably from about 50% to about 80%. However, one skilled in the art will appreciate that the compositions of the present invention can be made without any binders, e.g., to produce a highly friable dosage form.

In one aspect, the invention provides a solid composition comprising a diluent selected from the group consisting of microcrystalline cellulose and lactose.

The formulation further may comprise pH-adjusting agents. It is preferred to add such pH-adjusting acids to create and regulate a buffered microenvironment when combined with one or more alkalizers to obtain the desired delivery rate for the drug agent, Among those agents are but not limited to citric-acid, succinic acid, tartaric acid, acetic acid, vitamin C, and hydrochloric acid. Preferred are buffer substances like citric acid.

The pharmaceutical formulations disclosed herein can further comprise antioxidants and chelating agents. For example, the pharmaceutical formulations can comprise butylated hydroxyanisole (BHA), butylated hydroxytoluene (BHT), propyl gallate (PG), sodium metabisulfite, ascorbyl palmitate, potassium metabisulfite, disodium EDTA (ethylenediamine tetraacetic acid; also known as disodium edentate), EDTA, tartaric acid, citric acid, citric acid monohydrate, and sodium sulfite. In one embodiment, the foregoing compounds are included in the pharmaceutical formulations in amounts in the range of about 0.01% to about 5% w/w. In one specific embodiment, the pharmaceutical formulation includes BHA, BHT, or PG used at a range of about 0.02% to about 1% and disodium EDTA, citric acid, or citric acid monohydrate used at a range of about 2% to about 5%. In a preferred embodiment, the pharmaceutical formulation includes BHA used at about 0.05% w/w.

In one aspect, the invention provides a solid composition further comprising a plasticizer. The composition can also optionally include a plasticizer from about 0% to about 30% by weight, based on the total weight of the composition. In one embodiment, the plasticizer is from about 15% to about 25% by weight of the composition. Suitable plasticizers include, but are not limited to, triacetin, diethyl phthalate, tributyl sebacate, polyethylene glycol (PEG), glycerin, triacetin, and triaethyl citrate, for example. In one embodiment, the plasticizer is polyethylene glycol of molecular weight 200 to 20,000. In another embodiment, the plasticizer is polyethylene glycol of molecular weight 400 to 4,000. In another embodiment, the plasticizer is PEG 3350.

Lubricants can be used to prevent adhesion of the dosage form to the surface of the dies and punches, and to reduce inter-particle friction. Lubricants may also facilitate ejection of the dosage form from the die cavity and improve the rate of granulation flow during processing. Examples of suitable lubricants include, without limitation, magnesium stearate, glyceryl behenate, calcium stearate, zinc stearate, stearic acid, simethicone, silicon dioxide, talc, polyethylene glycol, mineral oil, carnauba wax, palmitic acid, sodium stearyl fumarate sodium laurel sulfate, glyceryl palmitostearate, myristic acid and hydrogenated vegetable oils and fats, as well as other known lubricants, and/or mixtures of two or more thereof. In one embodiment, the lubricant, if present, of the stock granulation is magnesium stearate. The compositions of the present invention can comprise from about 0% to about 10% by weight of the lubricant, and preferably from about 1% to about 5%.

In another embodiment, the composition can also optionally include an anti-adherent or glidant. Examples of glidants and/or anti-adherents suitable for use herein include but are not limited to, silicon dioxide, colloidal silicon dioxide, magnesium silicate, magnesium trisilicate, talc, and other forms of silicon dioxide, such as aggregated silicates and hydrated silica. In another embodiment, the composition can also optionally include an opacifying agent, such as titanium dioxide, for example. In yet another embodiment, the composition can also optionally include one or more colorants, for example, iron oxide based colorant(s).

The tablet composition may further comprise a protecting agent. The protecting agent coats at least part of the therapeutic agent, typically upon the mixing of the two agents. The protecting agent may be mixed with the therapeutic agent in a ratio from about 0.1 to about 100 by weight, preferably in a ratio from about 1 to about 50, and more preferably in a ratio of about 1 to about 10. Without being bound to any particular theory, the protecting agent reduces the adhesion between the therapeutic agent and the binder so that the therapeutic agent may be more easily released from the binder. In this way, the therapeutic agent may be delivered in the stomach within about 7 to about 12 hours, preferably within about 12 hours. Materials suitable as protecting agents are discussed in detail above and may be used alone or in combination in the tablet compositions of the present invention.

The tablet composition may also comprise one or more elastomeric solvents such as rosins and resins. Non-limiting examples of such solvents are discussed in detail above and may be used alone or in combination in the tablet compositions of the present invention. In addition, the tablet composition may further comprise waxes such as beeswax and microcrystalline wax, fats or oils such as soybean and cottonseed oil, and combinations thereof. Moreover, the tablet composition may additionally include plasticizers such as softeners or emulsifiers. Such plasticizers may, for example, help reduce the viscosity of the gastric solution of the dissolved tablet to a desirable consistency and improve its overall texture and bite and help facilitate the release of the therapeutic agent. Non-limiting examples of such plasticizers are discussed in detail above and may be used alone or in combination in the tablet compositions of the present invention.

In one embodiment of the stock granulation, the bulking agent is microcrystalline cellulose and/or lactose monohydrate, the binder, if present, is pregelatinized starch, the disintegrant, if present, is sodium starch glycolate, croscarmellose sodium and/or crospovidone, the lubricant, if present, is magnesium stearate and the glidant and/or anti-adherent, if present, is colloidal silicon dioxide and/or talc.

Sweetening agents can be used to improve the palatability of the composition by masking any unpleasant tastes it may have. Examples of suitable natural or artificial sweetening agents include, without limitation, compounds selected from the saccharide family such as the mono-, di-, tri-, poly-, and oligosaccharides; sugars such as sucrose, glucose (corn syrup), dextrose, invert sugar, fructose, maltodextrin, and polydextrose; saccharin and salts thereof such as sodium and calcium salts; cyclamic acid and salts thereof; dipeptide sweeteners; chlorinated sugar derivatives such as sucralose and dihydrochalcone; sugar alcohols such as sorbitol, sorbitol syrup, mannitol, xylitol, hexa-resorcinol, and the like, and combinations thereof. Hydrogenated starch hydrolysate, and the potassium, calcium, and sodium salts of 3,6-dihydro-6-methyl-1-1,2,3-oxathiazin-4-one-2,2-dioxide may also be used. The compositions of the present invention can comprise from about 0% to about 80% by weight of the sweetening agent, preferably from about 0.5% to about 75%, and more preferably from about 0.5% to about 50%.

Flavoring agents can also be used to improve the palatability of the composition. Examples of suitable flavoring agents include, without limitation, natural and/or synthetic (i.e., artificial) compounds such as peppermint, spearmint, wintergreen, cinnamon, menthol, cherry, strawberry, watermelon, grape, banana, peach, pineapple, apricot, pear, raspberry, lemon, grapefruit, orange, plum, apple, fruit punch, passion fruit, chocolate (e.g., white, milk, dark), vanilla, caramel, coffee, hazelnut, combinations thereof, and the like. Coloring agents can be used to color code the composition, for example, to indicate the type and dosage of the therapeutic agent therein. Suitable coloring agents include, without limitation, natural and/or artificial compounds such as FD & C coloring agents, natural juice concentrates, pigments such as titanium oxide, silicon dioxide, and zinc oxide, combinations thereof, and the like. The compositions of the present invention can comprise from about 0% to about 10% by weight of the flavoring and/or coloring agent, preferably from about 0.1% to about 5%, and more preferably from about 2% to about 3%.

Preparation of Solid Compositions Comprising Hydrophilic Polymer, Alkalizer and Active Agent into Tablets

Any suitable methods can be used to mix the formulation comprising the active agent, hydrophilic polymer and alkalizer. In one embodiment, the active agent, hydrophilic polymer and alkalizer are combined, mixed and the mixture may be directly compressed into a tablet. Typically, one or more vehicles or additives may be added to the mixture to improve flow and compressible characteristics. These additives include, for example, lubricants, such as magnesium stearate, zinc stearate, stearic acid, talc, and the like; flavors; and sweeteners. Direct compression has advantages, such as reducing cost, time, operational pace, and machinery; preventing active agent-excipient interaction; and less instability of active agent. Direct blending or dry granulation can also eliminate the possible pollution by organic solvent.

In another embodiment, some of the formulation components may be partially granulated prior to compression or all of the formulation components may be granulated prior to compression. For example, the active agent, alone can also be granulated prior to mixing. In another embodiment, the hydrophilic polymer (e.g., PEO) can be granulated prior to mixing with the active agent and/or with the alkalizer. In still yet another embodiment, the active agent can be granulated together with the hydrophilic polymer or the alkalizer, or all three together.

Any suitable granulation methods can be used to mix the formulation. In one embodiment, a wet granulation process can be used to mix one or more components of the formulation. For example, high shear granulation or fluid-bed granulation processes can be used. Any suitable commercially available granulation equipment can be used in these processes.

After the granulation of one or more components of the formulation, optionally, granulated formulation can be milled. Milling can be performed using any suitable commercially available apparatus, e.g., COMIL® equipped with a 0.039 inch screen. The mesh size for the screen of a COMIL® can be selected depending on the size of the granules desired. After wet granulated active agents are milled, they may be further dried (e.g., in a fluid-bed) if desired.

After preparing the formulation as described above, the formulation is compressed into a tablet form. This tablet shaping can be done by any suitable means, with or without compressive force. For example, compression of the formulation after the granulation step or blending can be accomplished using any tablet press, provided that the tablet composition is adequately lubricated unless an external lubrication process is used. The level of lubricant in the formulation is typically in the range of 0.5-2.0%, e.g. with magnesium stearate which is most commonly used as a lubricant. Many alternative means to effectuate this step are available, and the invention is not limited by the use of any particular equipment. The compression step can be carried out using a rotary type tablet press. The rotary type tableting machine has a rotary turret with multiple stations of dies and punches. The formulation is fed into the die and is subsequently compressed.

The tablet compositions can have any desired shape, size, and texture. The diameter and shape of the tablet depends on the molds, dies, and punches selected for the shaping or compression of the granulation composition. For example, tablets can be discoid, oval, oblong, round, cylindrical, triangular, and can have the shape of a stick, tab, pellet, sphere, and the like. Similarly, the tablet can be any desirable color. For example, the tablet can be any shade of red, blue, green, orange, yellow, violet, indigo, and mixtures thereof, and can be color coded to indicate the type and dosage of the therapeutic agent therein. The tablets may be scored to facilitate breaking. The top or lower surface can be embossed or debossed with a symbol or letters. The tablets can be individually wrapped or grouped together in pieces for packaging by methods well known in the art.

The compression force can be selected based on the type/model of press, what physical properties are desired for the tablets product (e.g., desired, hardness, friability, etc.), the desired tablet appearance and size, and the like. Typically, the compression force applied is such that the compressed tablets have a hardness of at least about 2 kP. These tablets generally provide sufficient hardness and strength to be packaged, shipped or handled by the user. If desired, a higher compression force can be applied to the tablet to increase the tablet hardness. However, the compression force is preferably selected so that it does not cause capping or lamination of tablets. Preferably, the compression force applied is such that the compressed tablet has a hardness of less than about 10 kP.

Typically, the final tablet will have a weight of about 50 mg to about 2000 mg, more typically about 200 mg to about 1000 mg, or about 400 mg to about 700 mg. In one aspect, the invention provides a solid composition wherein the amount of active agent is about 50 mg.

If desired, other modifications can be incorporated into embodiments of the invention. For example, modification of drug release through the tablet matrix of the present invention can also be achieved by any known technique, such as, e.g., application of various coatings, e.g., ion exchange complexes with, e.g., Amberlite IRP-69. The tablets of the invention can also include or be coadministered with GI motility-reducing drugs. Additional layers of coating can act as barriers for diffusion to provide additional means to control rate and timing of drug release.

In certain instances, the tablet composition includes a therapeutic agent centerfill. In addition, encapsulating the therapeutic agent in a centerfill may help to mask any undesirable taste that the therapeutic agent may have. In these instances, the binder surrounds, at least in part, a centerfill. The centerfill comprises at least one therapeutic agent, and may be a solid, liquid or semi-liquid material. The centerfill material can be a synthetic polymer, a semi-synthetic polymer, low-fat, or fat-free and contain one or more sweetening agents, flavoring agents, coloring agents, and/or scenting agents. Preferably, the centerfill includes a binary or ternary alkalizer as described herein.

In certain other instances, the tablet composition of the present invention is multilayered. In this way, the one or more therapeutic agents, e.g., two or more active agents or one or more active agents in combination with one or more non-active therapeutic agents can be delivered at defined dissolution rates. For example, with a bi-layered tablet, the first layer contains an active agent and the second layer contains the same or different active agent or a non-active therapeutic agent.

In still other instances, the combination of active agents with or without non-active therapeutic agents need not take the form of a multilayered tablet, but instead comprises a single homogenous tablet layer. This type of formulation may also be used in the case where gastrointestinal absorption of at least one therapeutic agent is desirable. In this case, the relative extent of ionization of the two or more therapeutic agents determines how they are to be absorbed.

The pharmaceutical formulations of the invention can be packaged in any packaging that facilitates stability of the drug formulation. For example, sealed high density polyethylene (HDPE) bottles containing silica gel desiccant or aluminum blister lined with PVC (thermoform PVC blister) or aluminum-aluminum blister can be used. Use of such packaging helps to control unwanted oxidation and moisture ingress of the product.

### Generation of a Predetermined Controlled Release Profile of the Active Agent

In one aspect, the invention provides a solid composition wherein the composition provides at least about 70% release of the active between about 7 to about 12 hours following oral administration. As such, the present invention provides methods for generating a predetermined controlled release profile of a pharmaceutically active agent. As described in the previous sections, the tablets of the invention comprise at least one pharmaceutically active agent, a hydrophilic polymer, and an alkalizer, the profile for the controlled release of the pharmaceutically active agent depends on factors such as the choice of the components of the hydrophilic polymer and alkalizer, their respective proportions, and whether any other material is included in the formulation. Thus, a desired release profile of a pharmaceutically active agent can be achieved by varying the kinds and levels of the hydrophilic polymer, and the alkalizer, e.g., the ratios of the hydrophilic polymer to the alkalizer by weight. By adding an optional component, the sustained release profile of the active agent can be further modified.

A more complex “programmable release profile,” which may comprise multiple stages in releasing active agent(s) with distinct release profile, can be achieved by combining layers of hydrophilic polymer with varying formulations, e.g., with varying percentages of one or more of the three main components of the formulation. In addition, the distribution pattern of the active agent blended within the hydrophilic polymer can contribute to the sustained release profile of the active agent from the tablet. When the particles are distributed in the hydrophilic polymer non-randomly (e.g., not evenly), a non-constant, but controlled level of active agent delivery can be achieved, such as, e.g., a pulsatile or delayed onset release profile. The tablets also can be designed and made such that “lag times” of release are incorporated into this scheme. For example, the tablets can be designed to have a delayed onset release of about 2 hours, about 3 hours, about 4 hours, about 5 hours, about 6 hours, or about 7 hours, after the administration.

In certain embodiments, the non-random drug distribution is controlled through a multilayer tablet formulation design and manufacturing process. Active agent distribution in the tablet is designed to be uneven (i.e., non-random). This can be achieved by manufacturing the tablet with multiple layers of formulation, with the layers having differing concentrations and/or types (e.g., modifications, pretreatments) of active agent. For example, alternative layers can have, in addition to varying amounts of active agent, particles comprising the same active agent by different amounts of coating materials or different compositions of coating materials, and the like, or varying amounts of any combination of these alternative forms. The layers can be of varying thickness. Moreover, one tablet can have one, two, three, four, fix, six, seven, eight, nine, ten, or any number of layers, limited only by the desired size of the finished tablet product, the thickness of each layer, the composition of each layer's formulation, the manufacturing process, and the like.

Various “pulsatile release” profiles can be designed by varying the rate at which the tablet dissolves as it passes through the digestive tract. This is accomplished by manufacturing different layers of the multilayered tablet with different kinds or amounts of active agent, hydrophilic polymer (e.g., PEO polymer or HPMC of varying molecular weights), alkalizer, different ratios of hydrophilic polymer to alkalizer, different percentages of other hydrophilic polymers if more than one type of hydrophilic polymer is used, different manufacturing compression forces, and the like. Alternatively, a compression coating process can also be employed. Thus, in addition to having different amounts or different modifications of active agent in each layer, the layers themselves can be pre-programmed to dissolve at different rates (and thus release active agent in different anatomical compartments) as the tablet passes through the digestive tract.

Whether the active agents are distributed randomly or non-randomly, a tablet can comprise one or more types of active agent, and/or one or more types of coating materials. The non-random distribution of active agent can be represented quantitatively by different amounts in different layers or qualitatively by having different forms of active agent in different layers, e.g., as having more coating materials in the particle in the outer layers as compared to the inner layers of the tablet, or, vice versa. In alternative embodiments, the non-random distribution of the active agent in the tablet is concentrated at the core of the tablet or is concentrated at the periphery of the tablet. In another embodiment, the tablet has multiple layers comprising varying amount of active agent or other formulation ingredients. Varying amounts of active agent can be in different layers of the multilayered tablet, e.g., increasing amounts of active agent in the outer layers as compared to the inner layers, or vice versa. Alternatively, different forms of active agent (e.g., encapsulated, granulated, conjugated) can be in different layers. Completely different types of active agents (e.g., drugs) or combinations thereof can be placed into different layers. The layers can be of varying thickness. One tablet can have one, two, three, four, five, six, seven, eight, nine, ten, or any number of layers, limited only by the desired size of the finished tablet product, the thickness of each layer, the composition of each layer's formulation, the manufacturing process, and the like.

The making of the varying layers of a multilayered or coated, controlled release tablet can be controlled through the compression coating process. A series of feeding devices equal in number to the number of layers to be designed in the tablet is distributed about a rotary disc (this scheme applies for both the direct compression and granulation processes). In operation, each feeding device emits a defined quantity of material into the female dies as the die travel by the feeding device's output valve. Each feeding device has a compressing device directly downstream, as seen in the direction of movement of the female dies. The compressing devices compress the material admitted into the female dies by the respective feeding devices. The compression causes the various layers of material to adhere to one another. Different amount of compressive force can be used for each layer.

When the desired number of layers has been formed, the resulting multilayered compressed tablet is ejected from the female die. Any appropriate apparatus for forming multilayer tablets can be used to make the pulsatile release tablets of the invention, e.g., powder layering in coating pans or rotary coaters; dry coating by double compression technique; wet or powder tablet coating by film coating technique, and the like. See, e.g., U.S. Pat. No. 5,322,655; Remington's Pharmaceutical Sciences Handbook: Chapter 90 “Coating of Pharmaceutical Dosage Forms”, 1990.

Different layers of the tablet can contain different amounts or kinds of formulation, including, e.g., PEO, HPMC, alkalizer, and/or active agent compositions. This variation in layers controls the amount and distribution of active agent within the tablet and its eventual release upon ingestion. The multilayered tablet can be further processed in any manner, e.g., by powder layering in coating pans or rotary coaters; dry coating by double compression technique, tablet coating by film coating technique, and the like.

### Methods of Administration

The compositions of the present invention are useful in therapeutic applications, e.g., for treating thrombosis. Importantly, the compositions of the present invention provide the rapid and predictable delivery of a active agent in the GI tract with surprisingly low inter-subject variability in terms of maximum plasma concentration (Cₘₐₓ) and the time to reach the maximum plasma concentration (Tₘₐₓ) by modulating the pH around the active. In particular, the delivery of the therapeutic agent optimizes absorption within the gastrointestinal tract. As a result, the therapeutic agent can reach the systemic circulation in a substantially shorter period of time and at a substantially higher concentration than with traditional oral (e.g., tablet) administration.

In addition, the compositions of the present invention offer advantages over compositions for oral administration that do not contain the hydrophilic polymer and alkalizer described herein. In particular, because the hydrophilic polymer and alkalizer in the compositions of the present invention can help increase the solubility of the active as pH increases up to pH 10 in a hydrate polymer matrix to enhance the product release profile, the therapeutic agent reaches the systemic circulation in a substantially shorter period of time (e.g., reducing the time to onset of therapeutic activity) and at a substantially higher concentration than with compositions for oral administration that do not contain the alkalizer.

The compositions of the present invention have particular utility in the area of human and veterinary therapeutics. Generally, administered dosages will be effective to deliver picomolar to micromolar concentrations of the active agent to the appropriate site.

Administration of the compositions of the present invention is preferably carried out via any of the accepted modes of solid-oral administration.

The following examples are intended for illustration only, are not intended to limit the scope of the invention. The contents of all U.S. patents and other references cited in this application are hereby incorporated by reference in the entirety.

### EXAMPLES

Various polymer matrix systems were evaluated for developing a once-daily controlled release tablet for Compound 1, which has poor aqueous solubility (˜<0.1 mg/ml at about 37° C.) and other weakly acidic drugs with similar properties (e.g. indomethacin, ketoprofen and naproxen). A series of formulations using various types of tablet matrices were made to control the drug release at a constant rate such that a minimum of about 70% of drug was released in about 7-9 hours (Fast Release: FR) and about 10-12 hours (Slow Release: SR). The hydrophilic polymers, including polyethylene oxide (PEO) and hydroxypropylmethylcellulose (HMPC); and alkalizers, including arginine HCl, calcium carbonate and magnesium oxide were utilized as the main formulation components for these controlled release dosage forms. Commonly used pharmaceutical excipients were used in the general formulations including: AVICEL® PH 102, Lactose Fastflo were used alone or in combination as a diluent in the formulations. Talc was used as a glidant and magnesium stearate used as a Lubricant in the formulations.

A wet granulation process was not used to make Compound 1 formulations as Compound 1 is moisture sensitive.

The packaging format used for packaging the core tablets for both formulations were 75 cc round white HDPE bottles with desiccant 2 gm canister and child resistant closure with induction seal.

Twenty five different formulations were made for a 50 mg Controlled Release (CR) tablet with a weight of 600-650 mg. The formulation batch size was approximately 50-100 tablets. The dosing strengths refer to the free acid quantity of Compound 1, potassium salt, indomethacin, ketoprofen or naproxen. The details of the formulations are summarized in the following Tables.

### Example 1

| Ingredient | % w/w |
|---|---|
| [4-(6-fluoro-7-methylamino-2,4-dioxo-1,4-dihydro-2H- | 8.25 |
| quinazolin-3-yl)-phenyl]-5-chloro-thiophen-2-yl- | |
| sulfonylurea potassium salt | |
| Microcrystalline cellulose (AVICEL ® PH 102) | 20.8 |
| Lactose fast flo | 26.44 |
| METHOCEL ™ K4M | 15.0 |
| Calcium carbonate | 20.0 |
| Magnesium oxide | 8.0 |
| Talc | 1.0 |
| Magnesium stearate | 0.5 |

### Example 2

| Ingredient | % w/w |
|---|---|
| [4-(6-fluoro-7-methylamino-2,4-dioxo-1,4-dihydro-2H- | 8.25 |
| quinazolin-3-yl)-phenyl]-5-chloro-thiophen-2-yl- | |
| sulfonylurea potassium salt | |
| Lactose fast flo | 23.25 |
| METHOCEL K4M | 26.00 |
| PEO polymer (POLYOX ™ WSR 1105) | 18.00 |
| Sodium bicarbonate | 20.00 |
| Citric acid monohydrate | 3.00 |
| Talc | 1.00 |
| Magnesium stearate | 0.5 |

### Example 3

| Ingredient | % w/w |
|---|---|
| [4-(6-fluoro-7-methylamino-2,4-dioxo-1,4-dihydro-2H- | 8.25 |
| quinazolin-3-yl)-phenyl]-5-chloro-thiophen-2-yl- | |
| sulfonylurea potassium salt | |
| Lactose fast flo | 34.25 |
| METHOCEL ™ K4M | 20.0 |
| PEO polymer (POLYOX ™ WSR 1105) | 18.0 |
| Sodium bicarbonate | 15.0 |
| Citric acid monohydrate | 3.0 |
| Talc | 1.0 |
| Magnesium stearate | 0.5 |

### Example 4

| Ingredient | % w/w |
|---|---|
| [4-(6-fluoro-7-methylamino-2,4-dioxo-1,4-dihydro-2H- | 8.25 |
| quinazolin-3-yl)-phenyl]-5-chloro-thiophen-2-yl- | |
| sulfonylurea potassium salt | |
| Microcrystalline cellulose (AVICEL ® PH 102) | 25.8 |
| Lactose fast flo | 30.44 |
| METHOCEL ™ K4M | 18.00 |
| METHOCEL ™ K100M | 1.00 |
| Arginine HCl | 15.00 |
| Talc | 1.00 |
| Magnesium stearate | 0.5 |

### Example 5

| Ingredient Name | % w/w |
|---|---|
| [4-(6-fluoro-7-methylamino-2,4-dioxo-1,4-dihydro-2H- | 8.25 |
| quinazolin-3-yl)-phenyl]-5-chloro-thiophen-2-yl- | |
| sulfonylurea potassium salt | |
| Microcrystalline cellulose (AVICEL ® PH 102) | 5.8 |
| Lactose fast flo | 19.45 |
| METHOCEL ™ K4M | 25.0 |
| Ethocel 20 | 10.0 |
| Calcium carbonate | 20.0 |
| Magnesium oxide | 10.0 |
| Talc | 1.0 |
| Magnesium stearate | 0.5 |

### Example 6

| Ingredient | % w/w |
|---|---|
| [4-(6-fluoro-7-methylamino-2,4-dioxo-1,4-dihydro-2H- | 8.25 |
| quinazolin-3-yl)-phenyl]-5-chloro-thiophen-2-yl- | |
| sulfonylurea potassium salt | |
| Microcrystalline Cellulose (AVICEL ® PH 102) | 25.24 |
| METHOCEL K4M | 25.0 |
| METHOCEL K100M | 5.0 |
| Calcium carbonate | 22.0 |
| Sodium bicarbonate | 5.0 |
| Magnesium oxide | 8.0 |
| Talc | 1.0 |
| Magnesium stearate | 0.5 |

### Example 7

| Ingredient | % w/w |
|---|---|
| [4-(6-fluoro-7-methylamino-2,4-dioxo-1,4-dihydro-2H- | 7.7 |
| quinazolin-3-yl)-phenyl]-5-chloro-thiophen-2-yl- | |
| sulfonylurea potassium salt | |
| Microcrystalline Cellulose (AVICEL ® PH 102) | 18.0 |
| Lactose fast flo | 29.8 |
| Carbopol 71G | 33.0 |
| Arginine HCl | 10.0 |
| Talc | 1.0 |
| Magnesium stearate | 0.5 |

### Example 8

| Ingredient | % w/w |
|---|---|
| [4-(6-fluoro-7-methylamino-2,4-dioxo-1,4-dihydro-2H- | 8.25 |
| quinazolin-3-yl)-phenyl]-5-chloro-thiophen-2-yl- | |
| sulfonylurea potassium salt | |
| Microcrystalline Cellulose (AVICEL ® PH 102) | 28.80 |
| Lactose fast flo | 24.44 |
| Glyceryl monostearate | 15.0 |
| Cetostearyl alcohol | 20.0 |
| PEG 3350 | 2.0 |
| Butylated hydroxyl anisole | 0.005 |
| Talc | 1.0 |
| Magnesium stearate | 0.5 |

### Example 9

| Ingredient | % w/w |
|---|---|
| [4-(6-fluoro-7-methylamino-2,4-dioxo-1,4-dihydro-2H- | 8.25 |
| quinazolin-3-yl)-phenyl]-5-chloro-thiophen-2-yl- | |
| sulfonylurea potassium salt | |
| Microcrystalline cellulose (AVICEL ® PH 102) | 20.8 |
| Lactose fast flo | 31.44 |
| METHOCEL ™ K4M | 10.0 |
| Calcium carbonate | 20.0 |
| Magnesium oxide | 8.0 |
| Talc | 1.0 |
| Magnesium stearate | 0.5 |

### Example 10

| Ingredient | % w/w |
|---|---|
| [4-(6-fluoro-7-methylamino-2,4-dioxo-1,4-dihydro-2H- | 8.25 |
| quinazolin-3-yl)-phenyl]-5-chloro-thiophen-2-yl- | |
| sulfonylurea potassium salt | |
| Microcrystalline cellulose (AVICEL ® PH 102) | 25.8 |
| Lactose fast flo | 34.44 |
| METHOCEL ™ K4M | 15.0 |
| Sodium phosphate dibasic anhydrous | 15.0 |
| Talc | 1.0 |
| Magnesium stearate | 0.5 |

### Example 11-13

| | Example 11 | Example 12 | Example 13 |
|---|---|---|---|
| Ingredient | % w/w | % w/w | % w/w |
| Indomethacin | 10.0 | 0.0 | 0.0 |
| Ketoprofen | 0.0 | 10.0 | 0.0 |
| Naproxen | 0.0 | 0.0 | 10.0 |
| Microcrystalline cellulose | 20.80 | 20.80 | 20.80 |
| (AVICEL ® PH 102) | | | |
| Lactose fast flo | 21.70 | 21.70 | 21.70 |
| METHOCEL ™ K4M | 15.00 | 15.00 | 15.00 |
| Calcium carbonate | 20.00 | 20.00 | 20.00 |
| Magnesium oxide | 8.00 | 8.00 | 8.00 |
| HPC EXF | 1.00 | 1.00 | 1.00 |
| Xanthan Gum | 2.00 | 2.00 | 2.00 |
| Talc | 1.0 | 1.0 | 1.0 |
| Magnesium stearate | 0.5 | 0.5 | 0.5 |

### Examples 14-16

| | Example 14 | Example 15 | Example 16 |
|---|---|---|---|
| Ingredient | % w/w | % w/w | % w/w |
| Indomethacin, | 10.0 | 0.0 | 0.0 |
| Ketoprofen | 0.0 | 10.0 | 0.0 |
| Naproxen | 0.0 | 0.0 | 10.0 |
| Microcrystalline cellulose | 20.80 | 20.80 | 20.80 |
| (AVICEL ® PH 102) | | | |
| Lactose fast flo | 21.70 | 21.70 | 21.70 |
| METHOCEL K4M | 15.00 | 15.00 | 15.00 |
| Calcium Phosphate Dibasic | 20.00 | 20.00 | 20.00 |
| Magnesium Oxide | 8.00 | 8.00 | 8.00 |
| HPC EXF | 1.00 | 1.00 | 1.00 |
| Xanthan Gum | 2.00 | 2.00 | 2.00 |
| Talc | 1.00 | 1.00 | 1.00 |
| Magnesium stearate | 0.50 | 0.5 | 0.5 |

### Example 17-19

| | Example 17 | Example 18 | Example 19 |
|---|---|---|---|
| Ingredient | % w/w | % w/w | % w/w |
| Indomethacin | 10.0 | 0.0 | 0.0 |
| Ketoprofen | 0.0 | 10.0 | 0.0 |
| Naproxen | 0.0 | 0.0 | 10.0 |
| Microcrystalline cellulose | 20.80 | 20.80 | 20.80 |
| (AVICEL ® PH 102) | | | |
| Lactose fast flo | 29.70 | 29.70 | 29.70 |
| PEO polymer (POLYOX ™ | 10.00 | 10.00 | 10.00 |
| WSR 1105) | | | |
| Calcium Carbonate | 20.00 | 20.00 | 20.00 |
| Magnesium Oxide | 8.00 | 8.00 | 8.00 |
| Talc | 1.00 | 1.00 | 1.00 |
| Magnesium stearate | 0.50 | 0.50 | 0.50 |

### Examples 20-22

| | Example 20 | Example 21 | Example 22 |
|---|---|---|---|
| Ingredient | % w/w | % w/w | % w/w |
| Indomethacin | 10.0 | 0.0 | 0.0 |
| Ketoprofen | 0.0 | 10.0 | 0.0 |
| Naproxen | 0.0 | 0.0 | 10.0 |
| PEO polymer (POLYOX ™ | 20.00 | 20.00 | 20.00 |
| WSR 1105) | | | |
| Lactose fast flo | 19.45 | 19.45 | 19.45 |
| METHOCEL ™ K4M | 27.80 | 27.80 | 27.80 |
| Calcium Phosphate Dibasic | 21.25 | 21.25 | 21.25 |
| Talc | 1.00 | 1.00 | 1.00 |
| Magnesium Stearate | 0.50 | 0.50 | 0.50 |

### Examples 23-25

| | Example 23 | Example 24 | Example 25 |
|---|---|---|---|
| Ingredient Name | % w/w | % w/w | % w/w |
| Indomethacin | 10.0 | 0.0 | 0.0 |
| Ketoprofen | 0.0 | 10.0 | 0.0 |
| Naproxen | 0.0 | 0.0 | 10.0 |
| Microcrystalline cellulose | 20.80 | 20.80 | 20.80 |
| (AVICEL ® PH 102) | | | |
| Lactose fast flo | 29.70 | 29.70 | 29.70 |
| PEO polymer (POLYOX ™ | 10.00 | 10.00 | 10.00 |
| WSR 1105) | | | |
| Calcium Phosphate Dibasic | 20.00 | 20.00 | 20.00 |
| Magnesium Oxide | 8.00 | 8.00 | 8.00 |
| Talc | 1.00 | 1.00 | 1.00 |
| Magnesium Stearate | 0.50 | 0.50 | 0.50 |

### Preparation

Tablets containing a suitable amount of an active ingredient (approximately 8 to 10% based on total weight of the tablet) were prepared by an appropriate method. For example, tablets containing 50 mg of [4-(6-fluoro-7-methylamino-2,4-dioxo-1,4-dihydro-2H-quinazolin-3-yl)-phenyl]-5-chloro-thiophen-2-yl-sulfonylurea acid (equivalent to 53.65 mg of potassium salt) are prepared using a direct compression method. The ingredients including the drug substance, alkalizers, polymers and binders are blended. Then the glidant and lubricant are mixed with the blend. This is followed by compression using a rotary tableting machine

### Dissolution Test Procedure

Dissolution tests were performed using a USP Apparatus 3 (VanKel Bio Dis or equivalent) with 36 vessels. Six individually weighted tablets were tested in an acid medium (250 mL of 0.5% Tween 80 in 0.1 N HCl) for 1 or 2 hours and then in a 50 mM sodium phosphate solution (pH=7.4; 250 mL) for up to 12 hours unless otherwise stated. The temperature of the dissolution media and the agitation rate were maintained at 37.0° C. (±0.5° C.) and 15 dpm, respectively. The concentration of the active agent in the samples collected at each time point were determined by reversed phase HPLC using a C₁₈, column (Thermo BDS Hypersil 5 μm, 150 mm×4.6 mm) and a UV detector at 248 nm

### Acid Robustness

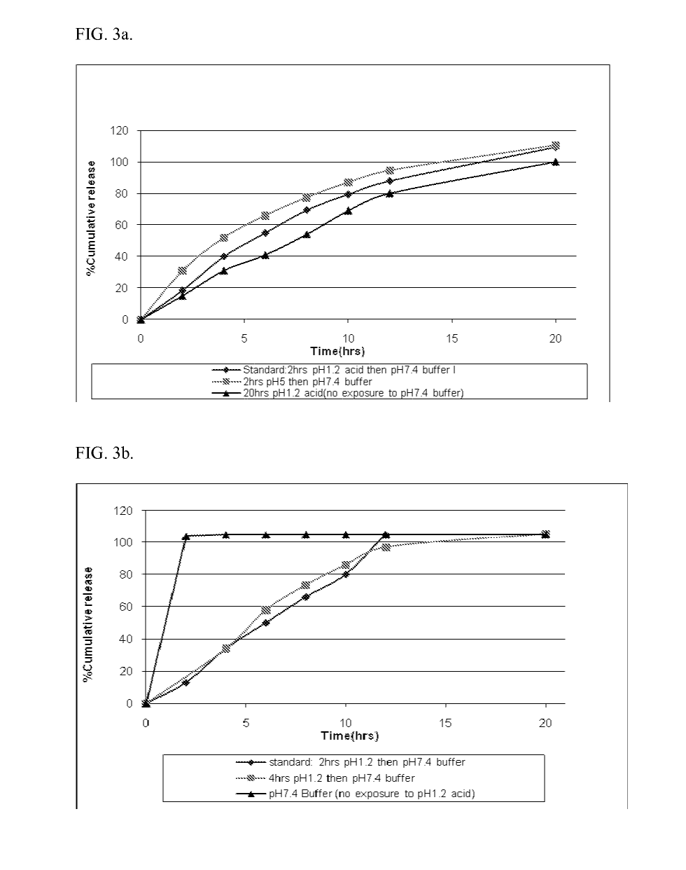

Dissolution profiles of Compound 1 tablets were collected under different pH conditions: (1) 2 hours in acidic (pH 1.2) medium before switching to a buffer (pH 7.4); (2) prolonged exposure in acid media for 4 hours prior to exposure to pH 7.4 buffer; and (3) in pH 7.4 buffer media, without prior exposure to acidic medium. The results for Example 1 and Example 2 are provided FIGS. 3A and 3B, respectively.

For Example 2, exposure in pH 7.4 buffer for 20 hours lead to 104% drug released in the first 2 hours. This may due to higher solubility of Compound 1 at pH 7.4 and it is dissolved before the rate controlling polymer is fully functional or hydrated. Exposure in pH 1.2 (acid) for 4 hours followed by pH 7.4 buffer or exposure in pH 5.0 buffer for 2 hours followed by pH 7.4 resulted in a dissolution profiles that were similar to those obtained with the standard dissolution media conditions (pH 1.2 (acid) for 2 hours followed by pH 7.4 buffer). As the physiological stomach pH ranges from about 1.2 to 5.0, the formulation is considered robust toward variation of pH in stomach environment.

Prolonged exposure in acid media for up to four hours followed by 7.4 pH buffer did not adversely affect the in-vitro release profile for the formulations tested.

### Formulation Stability

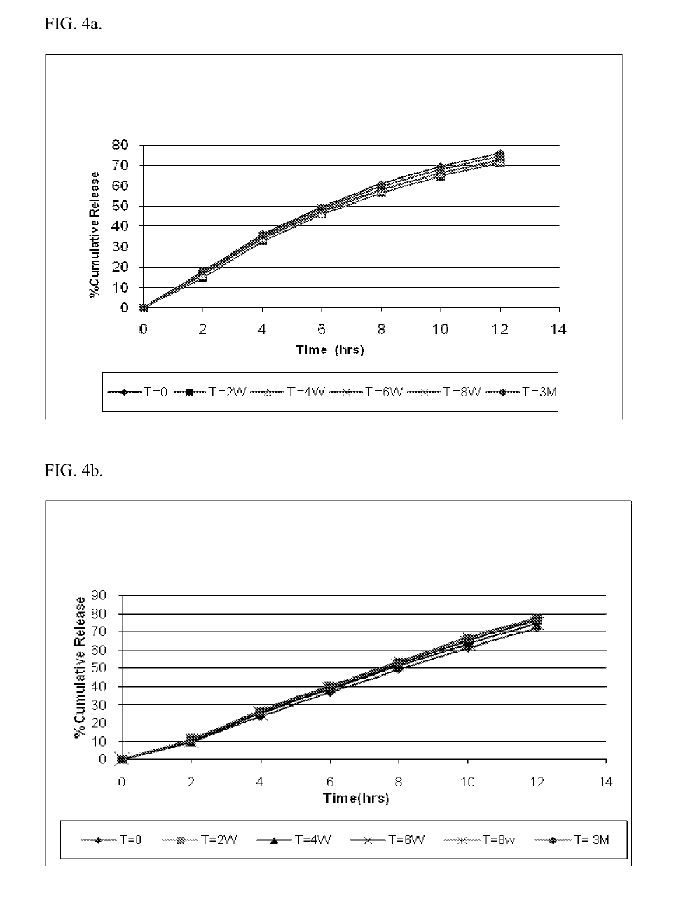

The stability results for dissolution are provided in FIGS. 4A and 4B for stability of Example 1 and 2.

The dissolution profiles, physical appearance, potency, related substance, moisture, and hardness were acceptable after storage at 40° C./75% RH up to the 3 months. The dissolution release profiles for the slow release formulations were also acceptable. In addition, the swollen tablet matrix remnant from dissolution was also analyzed for drug content and the results confirmed that the % recovery of between 90-110% of Compound 1 was achieved for all the formulations.

### Comparison of Dissolution Profiles for Direct Compression Vs Roller Compaction Formulations

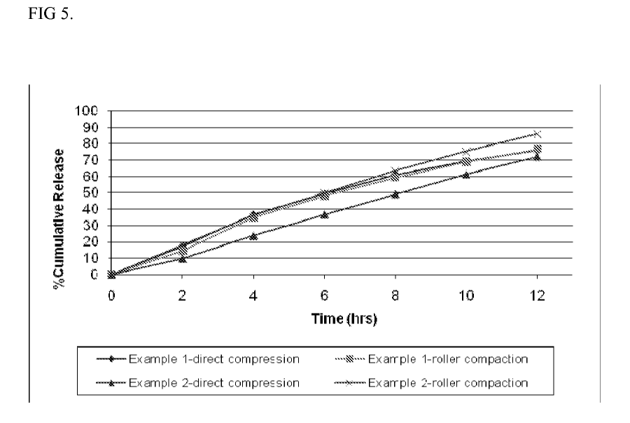

Similarly, Example 1 (SR) and Example 2 (FR) were made by the direct compression process and roller compression. Their comparative dissolution profiles are provided in FIG. 5.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A solid pharmaceutical composition for the controlled release of an active agent in the gastrointestinal tract, comprising:
(a) at least one acid active agent with a solubility of less than about 0.3 mg/ml in an aqueous solution at a pH of at most about the pKa of the acid active agent at a temperature of about 37° C., or a pharmaceutically acceptable salt thereof;
(b) at least one hydrophilic polymer; and
(c) at least one alkalizer;
wherein the composition reduces evacuation from the stomach; and
provides at least about 70% release of the active agent for a period of time from about between about 7 to about 12 hours following oral administration

2. The solid composition of claim 1, wherein the solubility of said active agent or a pharmaceutically acceptable salt thereof is less than about 0.2 mg/ml in an aqueous solution at a pH of at most about the pKa of the acid active agent at a temperature of about 37° C.

3. The solid composition of claim 1, wherein the solubility of said active agent or a pharmaceutically acceptable salt thereof is less than about 0.1 mg/ml in an aqueous solution at a pH of at most about the pKa of the acid active agent at a temperature of about 37° C.

4. The solid composition of claim 1, wherein the composition provides near zero order release profile independent of a pH range of about 1 to about 7.4.

5. The solid composition of claim 1, wherein the active agent has a solubility of less than about 0.1 mg/ml in an aqueous solution at a pH of about 1 to about 6.8.

6. The solid composition of claim 1, wherein the active agent has the formula (I): 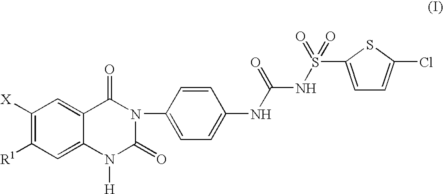
wherein:
R¹ is selected from the group consisting of H, halogen, —OH, —C₁₋₁₀-alkyl and C₁₋₆-alkylamino; and
X is selected from the group consisting of: F and I.

7. The solid composition of claim 1, wherein the active agent is [4-(6-fluoro-7-methylamino-2,4-dioxo-1,4-dihydro-2H-quinazolin-3-yl)-phenyl]-5-chloro-thiophen-2-yl-sulfonylurea potassium salt.

8. The solid composition of claim 1, wherein the amount of active agent is about 50 mg.

9. The solid composition of claim 1, wherein the amount of hydrophilic polymer is less than about 27.8% w/w of the composition.

10. The solid composition of claim 1, wherein the amount of hydrophilic polymer is between about 27.8% w/w to about 15 w/w % of the total composition.

11. The solid composition of claim 1, wherein the hydrophilic polymer has an average molecular weight of between about 0.82 and about 9×10⁵ Daltons.

12. The solid composition of claim 11, wherein the at least one hydrophilic polymer is a combination of hydrophilic polymers.

13. The solid composition of claim 1, wherein the hydrophilic polymer is selected from the group consisting of a cellulose ether, polyethylene oxide, acrylic acid, and combinations thereof.

14. The solid composition of claim 1, wherein the cellulose ether is METHOCEL™ K4M or K100M.

15. The solid composition of claim 1, wherein the polyethylene oxide is POLYOX™ WSR 1105.

16. The solid composition of claim 1, wherein the alkalizer selected from the group consisting of calcium carbonate, magnesium oxide, sodium bicarbonate and arginine and pharmaceutically acceptable salts thereof.

17. The solid composition of claim 1, wherein the total amount of alkalizer is from about 5 weight percent to about 50 weight percent of the total composition.

18. The solid composition of claim 1, wherein the total amount of alkalizer is from about 15 weight percent to about 30 weight percent of the total composition.

19. The solid composition of claim 18, wherein the combined weight percent of the alkalizer is greater than or equal to the weight percent of the active agent.

20. The solid composition of claim 1, wherein the weight ratio of said alkalizer to said hydrophilc polymer is from about 0.9 to about 0.69.

21. The solid composition of claim 1, wherein said composition comprises from about 7.6% w/w to about 8.9% w/w active agent; from about 27.8% w/w to about 15% w/w hydrophilic polymer; and from about 15% w/w to about 30% w/w alkalizer of the total composition.

22. The solid composition of claim 1, wherein the composition provides at least about 70% release of the active agent between about 7 to about 9 hours following oral administration.

23. The solid composition of claim 1, wherein the composition provides at least about 70% release of the active agent between about 10 to about 12 hours following oral administration.

24. The solid composition of claim 1, wherein the composition is a non-disintegrating matrix tablet.

25. The solid composition of claim 1, wherein the composition is a slow-disintegrating matrix tablet.

26. The solid composition of claim 1, further comprising a buffering system selected from at least one or combination of alkalizers and citric acid.

27. The solid composition of claim 1, wherein the composition is a floatation tablet.

28. A method for treating a cardiovascular disorder in a subject in need thereof, said method comprising:
administering to said subject a composition of claim 1.

29. The method of claim 28, wherein the cardiovascular disorder is thrombosis.

30. A method for producing a tablet, comprising:
(1) producing a mixture comprising:
(a) at least one weak acid active agent with a solubility of less than about 0.1 μg/ml in an aqueous solution at a pH of at most about the pKa of the active acid agent at a temperature of about 37° C., or a pharmaceutically acceptable salt thereof;
(b) at least one hydrophilic polymer which is not instantly soluble in gastric fluids; and
(c) an alkalizer;
wherein the composition reduces evacuation from the stomach; and
provides at least about 70% release of the active agent for a period of time from about between about 7 to about 12 hours following oral administration; and
(2) compressing the mixture to produce the tablet.
